(19) United States
(12) Patent Application Publication
(10) Pub. No.: US 2025/0361566 A1
(43) Pub. Date: Nov. 27, 2025

(54) TUMOR-INFORMED DIGITAL PCR PROFILING TECHNOLOGY FOR MONITORING CIRCULATING TUMOR DNA

(71) Applicant: ChromaCode Inc., Carlsbad, CA (US)

(72) Inventors: John ALVARADO, SANTA ANA, CA (US); Aaron AGUIAR, SAN DIEGO, CA (US); Lucien JACKY, ORANGE, CA (US); Dominic YURK, PASADENA, CA (US); Jerrod SCHWARTZ, CARLSBAD, CA (US)

(21) Appl. No.: 19/216,554

(22) Filed: May 22, 2025

Related U.S. Application Data
(60) Provisional application No. 63/650,801, filed on May 22, 2024.

(51) Int. Cl. C12Q 1/6886; C12Q 1/6851

(52) U.S. Cl. CPC C12Q 1/6886; C12Q 1/6851; C12Q 2600/156; C12Q 2600/16

(57) ABSTRACT

Described herein are Methods, systems, compositions, and macromolecule complexes, for detecting, analyzing, evaluating, screening for, prognosing, diagnosing, and/or monitoring, pre-cancerous and cancerous conditions with abnormal cell growth in a patient, including patients having Minimal Residual Disease (MRD).

### CROSS REFERENCE TO RELATED APPLICATION

This application claims the benefit under 35 U.S.C. § 119(e) of U.S. Provisional Application No. 63/650,801, filed May 22, 2024, the content of which is incorporated by reference in its entirety into the present disclosure.

### TECHNICAL FIELD

Methods, systems, compositions, and macromolecule complexes, for detecting, analyzing, evaluating, screening for, prognosing, diagnosing, and/or monitoring, pre-cancerous and cancerous conditions with abnormal cell growth in a patient.

### BACKGROUND

Methods, systems, compositions, and macromolecule complexes, for detecting, analyzing, evaluating, screening for, prognosing, diagnosing, and/or monitoring, pre-cancerous and cancerous conditions with abnormal cell growth in a patient are needed in biomedical research and in clinical settings. Because existing methods, systems, compositions, and macromolecule complexes are not optimal, there is a need in the field for improved methods, systems, and compositions.

### SUMMARY

Described herein are novel methods, systems, compositions, and macromolecule complexes, for detecting, analyzing, evaluating, screening for, prognosing, diagnosing, and/or monitoring, pre-cancerous and cancerous conditions with abnormal cell growth in a patient.

The novel composition disclosed herein is for use in a patient-specific PCR. In certain embodiments, the composition comprises one or more of the following components:
(a) a set of primers each encoding a sequence complementary to a unique patient-specific tumor variant sequence and
(b) a DNA polymerase; and/or
(c) a RNase H; and/or
(d) a dNTP mixture; and/or
(e) a buffer, and/or
(f) a magnesium compound.

In certain embodiments, the composition further comprises (g) a set of detection probes. In certain embodiments, each detection probe comprises a fluorophore and optionally a quencher. In certain embodiments, the fluorophore and optionally the quencher is conjugated to the probe. In certain embodiments, each detection probe encodes a sequence complementary to the prime. In certain embodiments, the number of unique detection probes for each unique patient-specific tumor variant sequence is between 1 and np, wherein np is selected from the list consisting of 1, 2, 3, 4, 5, 6, 7, 8, 9, 10, 11, and 12. In certain embodiments, the number of unique detection probes specific for the unique patient-specific tumor variant sequences in a sample volume is between 1 and Np, wherein Np is selected from the list of 1, 4, 8, 12, 16, 20, 24, 28, 32, 36, 40, 44, 48, 52, 56, 60, 64, 68, 72, 76, 80, 84, 88, 92 and 96 or 2, 3, 4, 5, 6, 7, 8, 9, 10, 11, 12, 13, 14, 15, 16, 17, 18, 19, and 20. In certain embodiments, the first detection probe is conjugated to a first fluorophore and optionally conjugated to a first quencher. In certain embodiments, the combination of the emission color and emission intensity for each detection probe is unique. In certain embodiments, the fluorophore is includes ABY, Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750,ATTO 425, ATTO 550, ATTO 590, Cyan500, Cy3, Cy5, Cy5.5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, VIC, LC610, CFR610, JA270, LC640, JUN, Trimethylrhodamine (TRITC), Cal Fluor dyes, Quasar dyes, DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), derivatives thereof and combinations thereof. In certain embodiments, the fluorophore is selected from a group consisting of ATTO 425, FAM, HEX, TAMRA, Texas Red, Cy5, ATTO 590, ROX, or Cy5.5, derivatives thereof, and combinations thereof. In certain embodiments, the fluorophore is selected from a group of ATTO 425, FAM, HEX, Texas Red, Cy5, Cy5.5, derivatives thereof, and combinations thereof. In certain embodiments, the quencher can be TAMRA, BHQ-1, BHQ-2, BHQ-3, IowaBlack FQ, ZEN, or Dabcy, derivatives thereof, and combinations thereof.

In certain embodiments, the set of primers each encode a set of tag sequences.

In certain embodiments, the number of unique tag sequence on any individual primer is between 1 and n, wherein n is selected from the list consisting of 1, 2, 3, 4, 5, 6, 7, 8, 9, 10, 11, and 12. In certain embodiments, the total number of unique tag sequences on the set of primers is between 1 and N, wherein N is selected from the list consisting of 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 80, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536.

In certain embodiments, the total number of unique primers in the set of primers is between 1 and X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, 1536, 1540, 1544, 1548, 1552, 1556, 1560, 1564, 1568, 1572, 1576, 1580, 1584, 1588, 1592, 1596, 1600, 1604, 1608, 1612, 1616, 1620, 1624, 1628, 1632, 1636, 1640, 1644, 1648, 1652, 1656, 1660, 1664, 1668, 1672, 1676, 1680, 1684, 1688, 1692, 1696, 1700, 1704, 1708, 1712, 1716, 1720, 1724, 1728, 1732, 1736, 1740, 1744, 1748, 1752, 1756, 1760, 1764, 1768, 1772, 1776, 1780, 1784, 1788, 1792, 1796, 1800, 1804, 1808, 1812, 1816, 1820, 1824, 1828, 1832, 1836, 1840, 1844, 1848, 1852, 1856, 1860, 1864, 1868, 1872, 1876, 1880, 1884, 1888, 1892, 1896, 1900, 1904, 1908, 1912, 1916, 1920, 1924, 1928, 1932, 1936, 1940, 1944, 1948, 1952, 1956, 1960, 1964, 1968, 1972, 1976, 1980, 1984, 1988, 1992, 1996, 2000, 2004, 2008, 2012, 2016, 2020, 2024, 2028, 2032, 2036, 2040, 2044, 2048, 2052, 2056, 2060, 2064, 2068, 2072, 2076, 2080, 2084, 2088, 2092, 2096, 2100, 2104, 2108, 2112, 2116, 2120, 2124, 2128, 2132, 2136, 2140, 2144, 2148, 2152, 2156, 2160, 2164, 2168, 2172, 2176, 2180, 2184, 2188, 2192, 2196, 2200, 2204, 2208, 2212, 2216, 2220, 2224, 2228, 2232, 2236, 2240, 2244, 2248, 2252, 2256, 2260, 2264, 2268, 2272, 2276, 2280, 2284, 2288, 2292, 2296, 2300, 2304, 2308, 2312, 2316, 2320, 2324, 2328, 2332, 2336, 2340, 2344, 2348, 2352, 2356, 2360, 2364, 2368, 2372, 2376, 2380, 2384, 2388, 2392, 2396, 2400, 2404, 2408, 2412, 2416, 2420, 2424, 2428, 2432, 2436, 2440, 2444, 2448, 2452, 2456, 2460, 2464, 2468, 2472, 2476, 2480, 2484, 2488, 2492, 2496, 2500, 2504, 2508, 2512, 2516, 2520, 2524, 2528, 2532, 2536, 2540, 2544, 2548, 2552, 2556, 2560, 2564, 2568, 2572, 2576, 2580, 2584, 2588, 2592, 2596, 2600, 2604, 2608, 2612, 2616, 2620, 2624, 2628, 2632, 2636, 2640, 2644, 2648, 2652, 2656, 2660, 2664, 2668, 2672, 2676, 2680, 2684, 2688, 2692, 2696, 2700, 2704, 2708, 2712, 2716, 2720, 2724, 2728, 2732, 2736, 2740, 2744, 2748, 2752, 2756, 2760, 2764, 2768, 2772, 2776, 2780, 2784, 2788, 2792, 2796, 2800, 2804, 2808, 2812, 2816, 2820, 2824, 2828, 2832, 2836, 2840, 2844, 2848, 2852, 2856, 2860, 2864, 2868, 2872, 2876, 2880, 2884, 2888, 2892, 2896, 2900, 2904, 2908, 2912, 2916, 2920, 2924, 2928, 2932, 2936, 2940, 2944, 2948, 2952, 2956, 2960, 2964, 2968, 2972, 2976, 2980, 2984, 2988, 2992, 2996, 3000, 3004, 3008, 3012, 3016, 3020, 3024, 3028, 3032, 3036, 3040, 3044, 3048, 3052, 3056, 3060, 3064, 3068, and 3072.

In certain embodiments, the number of unique patient-specific tumor variant sequence is from 1 to X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536.

In certain embodiments, the tumor variants correlate with or is associated with a lesion, benign tumor, pre-malignant tumor, malignant tumor, neoplasia, dysplasia, hyperplasia, hamartoma, and/or other pre-cancerous and cancerous conditions with abnormal cell growth in the patient. In certain embodiments, the tumor variants sequence is different from the corresponding sequence of a normal somatic cell in the patient. In certain embodiments, the tumor variants sequence is preferably listed in a private and/or public database. In certain embodiments, the tumor variants sequence is listed in the public database. In certain embodiments, the public database comprises COSMIC, ClinVar, OncoKB, and/or combinations thereof.

The novel composition disclosed herein complex described herein is for use in detecting, analyzing, evaluating, screening for, prognosing, diagnosing, and/or monitoring, a condition in a patient, the complex comprising an amplicon, a primer, and a set of detection probes. In certain embodiments, the amplicon encodes a patient-specific tumor variant sequence. In certain embodiments, the primer encodes a sequence complementary to the unique patient-specific tumor variant sequence, and a set of tag sequences described elsewhere in this Application.

In certain embodiments, each detection probe comprises a fluorophore. In certain embodiments, each detection probe comprises a quencher. In certain embodiments, the fluorophore and optionally the quencher is conjugated to the probe. In certain embodiments, each detection probe encodes a sequence complementary to the primer.

In certain embodiments, the number of unique detection probes on the complex is between 1 and np, wherein np is selected from the list consisting of 1, 2, 3, 4, 5, 6, 7, 8, 9, 10, 11, and 12. In certain embodiments, the primer is configured to anneal on the amplicon. In certain embodiments, the detecting the complex is indicative of the condition in the patient. In certain embodiments, the detecting the complex detects a plurality of complexes each with a unique tumor variant sequence. In certain embodiments, the number of unique patient-specific tumor variants is from 1 to X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 768, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536.

In certain embodiments, the number of unique detection probes in the plurality of complexes is between 1 and Np. In certain embodiments, Np is selected from the list consisting of 1, 4, 8, 12, 16, 20, 24, 28, 32, 36, 40, 44, 48, 52, 56, 60, 64, 68, 72, 76, 80, 84, 88, 92 and 96. In certain embodiments, Np is selected from the list consisting of 2, 3, 4, 5, 6, 7, 8, 9, 10, 11, 12, 13, 14, 15, 16, 17, 18, 19, and 20.

In certain embodiments, the first detection probe is conjugated to a first fluorophore. In certain embodiments, the first detection probe is conjugated to a first quencher. In certain embodiments, the combination of the emission color and emission intensity for each detection probe is unique.

In certain embodiments, the fluorophore is selected from a group consisting of ABY, Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750, ATTO 425, ATTO 550, ATTO 590, Cyan500, Cy3, Cy5, Cy5.5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, VIC, LC610, CFR610, JA270, LC640, JUN, Trimethylrhodamine (TRITC), Cal Fluor dyes, Quasar dyes, DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), a derivative thereof and a combination thereof. In certain embodiments, the fluorophore is selected from a group consisting of ATTO 425, FAM, HEX, TAMRA, Texas Red, Cy5, ATTO 590, ROX, Cy5.5, a derivative thereof, and a combination thereof. In certain embodiments, the fluorophore is selected from a group consisting of ATTO 425, FAM, HEX, Texas Red, Cy5, Cy5.5, a derivative thereof, and a combination thereof.

In certain embodiments, the quencher is selected from the group consisting of TAMRA, BHQ-1, BHQ-2, BHQ-3, lowaBlack FQ, ZEN, Dabcy, a derivative thereof, and a combination thereof.

In certain embodiments, the composition comprises a fluorophore and optionally a quencher, and a DNA probe sequence. In certain embodiments, the DNA probe sequence comprise 11 to 30 nucleotide bases. In certain embodiments, the DNA probe sequence exhibiting a melting point between 45° C. and 75° C. In certain embodiments, the sequence is either unmodified or modified to achieve a melting point between 65° C. and 75° C. In certain embodiments, the melting point is achieved by including one or more locked nucleic acid (LNA) bases. In certain embodiments, the melting point is achieved by including one or more peptide nucleic acid (PNA) bases. In certain embodiments, the melting point is achieved by including one or more 2′-O-methyl RNA nucleotides. In certain embodiments, the melting point is achieved by including one or more phosphorothioate (PS) linkage modifications. In certain embodiments, the melting point is achieved by further conjugating with minor groove binding (MGB) proteins.

In certain embodiments, the condition as described herein is lesion, benign tumor, pre-malignant tumor, malignant tumor, neoplasia, dysplasia, hyperplasia, hamartoma, and/or other pre-cancerous and cancerous conditions with abnormal cell growth. In certain embodiments, the condition as described herein is a relapse of a cancer. In certain embodiments, the condition as described herein is a solid tumor. In certain embodiments, the condition as described herein is a hematologic malignancy.

In certain embodiments, the condition as described herein is a Minimal Residual Disease (MRD). In certain embodiments, the MRD is a breast cancer; colorectal cancer; lung cancer, including non-small cell lung cancer (NSCLC) and/or small cell lung cancer (SCLC); melanoma; bladder cancer; ovarian cancer; gastric cancer; prostate cancer; pancreatic cancer; esophageal cancer; head and neck cancer; glioblastoma; sarcoma; thyroid cancer; renal cell carcinoma; hepatocellular carcinoma; cervical cancer; endometrial cancer; testicular cancer; neuroblastoma, and/or combinations thereof. In certain embodiments, the MRD is a leukemia, preferably acute lymphoblastic leukemia (ALL), chronic lymphocytic leukemia (CLL), chronic myeloid leukemia (CML), and/or acute myeloid leukemia (AML); lymphoma, preferably non-Hodgkin's lymphoma (NHL), Hodgkin's lymphoma; follicular lymphoma, mantle cell lymphoma, T-cell lymphomas, precursor B-cell lymphoblastic lymphoma, diffuse large B-cell lymphoma (DLBCL), and/or Burkitt lymphoma; Waldenström's macroglobulinemia, multiple myeloma, myelodysplastic syndromes (MDS), and/or combinations thereof.

In certain embodiments, the tumor variants correlate with or is associated with a lesion, benign tumor, pre-malignant tumor, malignant tumor, neoplasia, dysplasia, hyperplasia, hamartoma, and/or other pre-cancerous and cancerous conditions with abnormal cell growth in the patient. In certain embodiments, the tumor variant sequence is different from the corresponding sequence of a normal somatic cell in the patient. In certain embodiments, the tumor variant sequence is listed in a private and/or public database. In certain embodiments, the public database comprises COSMIC, ClinVar, OncoKB, and/or combinations thereof.

The novel mixture for use in patient-specific PCR disclosed herein comprising a plurality of the novel complex disclosed herein. In certain embodiments, the mixture comprises:
(a) a set of primers each encoding a sequence complementary to a unique patient-specific tumor variant sequence and
(b) a DNA polymerase; and/or
(c) a RNase H; and/or
(d) a dNTP mixture; and/or
(e) a buffer, and/or
(f) a magnesium compound.

In certain embodiments, the set of primers each encode a set of tag sequences. In certain embodiments, the total number of unique tag sequences on the set of primers is between 1 and N. In certain embodiments, N is selected from the list consisting of 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536. In certain embodiments, the number of unique patient-specific tumor variant sequence is from 1 to n. In certain embodiments, the total number of unique primers in the set of primers is between 1 and X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, 1536, 1540, 1544, 1548, 1552, 1556, 1560, 1564, 1568, 1572, 1576, 1580, 1584, 1588, 1592, 1596, 1600, 1604, 1608, 1612, 1616, 1620, 1624, 1628, 1632, 1636, 1640, 1644, 1648, 1652, 1656, 1660, 1664, 1668, 1672, 1676, 1680, 1684, 1688, 1692, 1696, 1700, 1704, 1708, 1712, 1716, 1720, 1724, 1728, 1732, 1736, 1740, 1744, 1748, 1752, 1756, 1760, 1764, 1768, 1772, 1776, 1780, 1784, 1788, 1792, 1796, 1800, 1804, 1808, 1812, 1816, 1820, 1824, 1828, 1832, 1836, 1840, 1844, 1848, 1852, 1856, 1860, 1864, 1868, 1872, 1876, 1880, 1884, 1888, 1892, 1896, 1900, 1904, 1908, 1912, 1916, 1920, 1924, 1928, 1932, 1936, 1940, 1944, 1948, 1952, 1956, 1960, 1964, 1968, 1972, 1976, 1980, 1984, 1988, 1992, 1996, 2000, 2004, 2008, 2012, 2016, 2020, 2024, 2028, 2032, 2036, 2040, 2044, 2048, 2052, 2056, 2060, 2064, 2068, 2072, 2076, 2080, 2084, 2088, 2092, 2096, 2100, 2104, 2108, 2112, 2116, 2120, 2124, 2128, 2132, 2136, 2140, 2144, 2148, 2152, 2156, 2160, 2164, 2168, 2172, 2176, 2180, 2184, 2188, 2192, 2196, 2200, 2204, 2208, 2212, 2216, 2220, 2224, 2228, 2232, 2236, 2240, 2244, 2248, 2252, 2256, 2260, 2264, 2268, 2272, 2276, 2280, 2284, 2288, 2292, 2296, 2300, 2304, 2308, 2312, 2316, 2320, 2324, 2328, 2332, 2336, 2340, 2344, 2348, 2352, 2356, 2360, 2364, 2368, 2372, 2376, 2380, 2384, 2388, 2392, 2396, 2400, 2404, 2408, 2412, 2416, 2420, 2424, 2428, 2432, 2436, 2440, 2444, 2448, 2452, 2456, 2460, 2464, 2468, 2472, 2476, 2480, 2484, 2488, 2492, 2496, 2500, 2504, 2508, 2512, 2516, 2520, 2524, 2528, 2532, 2536, 2540, 2544, 2548, 2552, 2556, 2560, 2564, 2568, 2572, 2576, 2580, 2584, 2588, 2592, 2596, 2600, 2604, 2608, 2612, 2616, 2620, 2624, 2628, 2632, 2636, 2640, 2644, 2648, 2652, 2656, 2660, 2664, 2668, 2672, 2676, 2680, 2684, 2688, 2692, 2696, 2700, 2704, 2708, 2712, 2716, 2720, 2724, 2728, 2732, 2736, 2740, 2744, 2748, 2752, 2756, 2760, 2764, 2768, 2772, 2776, 2780, 2784, 2788, 2792, 2796, 2800, 2804, 2808, 2812, 2816, 2820, 2824, 2828, 2832, 2836, 2840, 2844, 2848, 2852, 2856, 2860, 2864, 2868, 2872, 2876, 2880, 2884, 2888, 2892, 2896, 2900, 2904, 2908, 2912, 2916, 2920, 2924, 2928, 2932, 2936, 2940, 2944, 2948, 2952, 2956, 2960, 2964, 2968, 2972, 2976, 2980, 2984, 2988, 2992, 2996, 3000, 3004, 3008, 3012, 3016, 3020, 3024, 3028, 3032, 3036, 3040, 3044, 3048, 3052, 3056, 3060, 3064, 3068, and 3072.

Described herein is a novel composition comprising a plurality of detection probes. In certain embodiments, each detection probe comprises a fluorophore. In certain embodiments, each detection probe comprises a quencher. In certain embodiments, each detection probe encodes a sequence complementary to a synthetic sequence encoded by a primer. In certain embodiments, composition emit a unique signal in the sequence encoded by the primer.

Described herein is a novel method of making the composition for patient-specific PCR. The method comprising:
(a) isolating
   a first genomic DNA from a first sample of a patient and
   a second genomic DNA from a second sample of the patient,
(b) sequencing
   the first genomic DNA and
   the second genomic DNA,
(c) identifying a set of the unique patient-specific tumor variant sequences
(d) synthesizing the set of primers for the patient-specific tumor variant sequences identified in step (c), and
(e) combining the set of primers with the set of reagents to form the composition for patient-specific PCR.

In certain embodiments, the patient-specific PCR is used to detect, analyze, evaluate, screen for, prognose, diagnose, and/or monitor, a condition in the patient. In certain embodiments, the condition is a lesion, benign tumor, pre-malignant tumor, malignant tumor, neoplasia, dysplasia, hyperplasia, hamartoma, and/or other pre-cancerous and cancerous conditions with abnormal cell growth.

In certain embodiments, the first sample comprises a cancerous tissue biopsy. In certain embodiments, the tissue biopsy is suspected of being cancerous. In certain embodiments, the second sample comprises a normal or non-cancerous blood sample. In certain embodiments, the normal or non-cancerous blood sample is a normal buffy coat of the blood sample. In certain embodiments, the second sample comprises a normal or non-cancerous tissue biopsy. In certain embodiments, the first genomic DNA comprise a DNA that is cancerous. In certain embodiments, the DNA that is suspected of being cancerous. In certain embodiments, the second genomic DNA comprises a normal DNA. In certain embodiments, the normal DNA is isolated from a leukocyte or a buffy coat of the second sample.

In certain embodiments, the sequencing of step (b) is not whole genome sequencing. In certain embodiments, the sequencing of step (b) comprises exome sequencing. In certain embodiments, the sequencing of step (b) comprises deep targeted sequencing. In certain embodiments, the sequencing of step (b) comprises shearing the genomic DNA from the first sample into fragments having a length of from approximately 2 to 2000 nucleotides, from 2 to 4000 nucleotides, from 2 to 10,000 nucleotides. In certain embodiments, the sequencing of step (b) comprises sheering the genomic DNA from the second sample into fragments having a length of from approximately 2 to 2000 nucleotides, from 2 to 4000 nucleotides, from 2 to 10,000 nucleotides. In certain embodiments, the sequencing of step (b) comprises performing an end-repair and A-tailing step. In certain embodiments, the sequencing of step (b) comprises ligating a plurality of synthetic adapters to the A-tailed genomic DNA of the first sample and/or the second sample. In certain embodiments, the sequencing of step (b) comprises hybridizing a biotinylated RNA bait or probe complementary to a region on the exome of the genomic DNA of the first sample and/or the second sample. In certain embodiments, the sequencing of step (b) comprises using a streptavidin bead to isolate the hybridized genomic DNA from the first sample and/or the second sample. In certain embodiments, the sequencing of step (b) comprises washing the streptavidin bead. In certain embodiments, the sequencing of step (b) comprises digesting or removing any RNA. In certain embodiments, the sequencing of step (b) comprises enriching the genomic DNA from the first sample and/or the second sample in a PCR using two primers targeting the synthetic ligated-in adapters.

In certain embodiments, step (c) comprises identifying a first broad set of variant sequences by comparing the sequences of the first genomic DNA and the second genomic DNA. In certain embodiments, step (c) comprises identifying a second narrower set of variant sequences by comparing the sequences of the first genomic DNA and the second genomic DNA with known tumor variants. In certain embodiments, the known tumor variants are listed in a private and/or public database. In certain embodiments, the public database is selected from COSMIC, ClinVar, OncoKB, and combinations thereof. In certain embodiments, step (c) comprises identifying a third prioritized set of variant sequences using a set of selection criteria comprised of a tumor variant allele frequency, a tumor variant allele population prevalence, tumor variant driver status, a sequence context, a PCR and/or dPCR detection suitability, and/or a PCR or dPCR compatibility with the other selected variant sequences.

In certain embodiments, in the primer of step (d), the annealing temperature of each primer and the complementary sequence is less than the melting temperature of the primer and the complementary sequence. In certain embodiments, in the primer of step (d), the difference between median melting temperature of the primer set and annealing temperature is selected from the list consisting of from about 5° C. to about 7° C., from about 2° C. to about 5° C., from about 1° C. to about 4° C., from about 3° C. to about 6° C. and from about 6° C. to about 12° C. In certain embodiments, in the primer of step (d), the set of primers is capable of detecting a portion of the patient-specific tumor variant sequences in step (c), preferably more than 30% of the patient-specific tumor variant sequences, more preferably more than 50% of the patient-specific tumor variant sequences, more preferably more than 60% of the patient-specific tumor variant sequences, more preferably more than 70% of the patient-specific tumor variant sequences, more preferably more than 80% of the patient-specific tumor variant sequences, more preferably more than 90% of the patient-specific tumor variant sequences, more preferably more than 95% of the patient-specific tumor variant sequences, and more preferably more than 100% of the patient-specific tumor variant sequences.

In certain embodiments, the set of reagents comprise (1) a plurality of detection probes encoding sequences complementary to the set of tag sequences. In certain embodiments, the number of unique detection probes on the complex is between 1 to np, wherein np is selected from the list consisting of 1, 2, 3, 4, 5, 6, 7, 8, 9, 10, 11, and 12. In certain embodiments, the first detection probe is conjugated to a first fluorophore and optionally conjugated to a first quencher. In certain embodiments, the next detection probe(s) is conjugated to a next fluorophore. In certain embodiments, the next detection probe(s) is conjugated to a next quencher, and the nth detection probe is conjugated to a nth fluorophore and optionally conjugated to an nth quencher. In certain embodiments, combination of the emission color and intensity of each fluorophore or fluorophore and quencher combination in the plurality of detection probes is unique. In certain embodiments, the reagents further comprise: (2) a DNA polymerase, (3) a RNase H, (4) a dNTP mixture, (5) a buffer, and/or (6) a magnesium compound. In certain embodiments, one or more of the reagents (2)-(6) are stored in separate compartments.

The novel method for detecting, analyzing, evaluating, screening for, prognosing, diagnosing, and/or monitoring, a condition in the patient disclosed herein comprises:
(1) periodically collecting blood samples from the patient;
(2) isolating cell-free DNA from the patient's (a) plasma;
(3) isolating genomic DNA from the patient's leukocytes and/or buffy coat;
(4) performing a digital PCR using the patient-specific primers, and
(5) detecting the presence of key tumor-specific variants based on the individually unique primer tags designed-in the patent specific digital PCR mastermix(es) disclosed elsewhere in this Application. In certain embodiments, step (1) is collected from the patient at 1 week to 12 months. In certain embodiments, the method does not involve sequencing the patient cell-free DNA of step (2) and in certain embodiments, the method does not involve sequencing genomic DNA of step (3). In certain embodiments, the method includes conducting a quality control test on the yield and/or size of the cell-free DNA of step (2) and genomic DNA of step (3). In certain embodiments, the method includes performing a pre-amplification of the cell-free DNA sample for regions containing patient-specific tumor variant sequence identified. In certain embodiments, the method includes splitting the pre-amplified material between a plurality of parallel amplification reactions. In certain embodiments, the plurality of parallel amplification reaction is from 1 to about 16. In certain embodiments, the method includes performing digital PCR using the bespoke patient-specific digital PCR mastermixes to measure tumor-specific variants on the cell-free DNA and genomic DNA. In certain embodiments, the method includes automatically analyzing digital PCR data in cloud software to determine:
(a) the tumor-specific variants present, if any, and/or
(b) the copy numbers of the tumor-specific variants in the sample, and/or
(c) allele frequency of the tumor-specific variants.
   In certain embodiments, the method includes generating a report that includes:
(a) a description of the tumor-specific variants present, if any;
(b) an assessment of overall tumor load in circulation, and
(c) a description of circulating tumor load trajectory information based on the longitudinal series of timepoints.

The novel for kit for detecting, analyzing, evaluating, screening for, prognosing, diagnosing, and/or monitoring, a condition in the patient as disclosed herein comprises the composition as described herein and/or the mixture described herein, and an instruction for the method described herein. In certain embodiments, one or more of (a)-(g) of the composition described herein are stored separately in a container. In certain embodiments, two or more of (a)-(g) of the composition described herein are pre-mixed and stored together in a container. In certain embodiments, one or more of (a)-(f) of the mixture described herein are stored separately in a container. In certain embodiments, two or more of (a)-(f) of the mixture described herein are pre-mixed and stored together in a container.

### BRIEF DESCRIPTION OF THE DRAWINGS

FIG. 1 shows a schematic of the workflow.

FIG. 2 shows an example schematic of 8 target sequences and 8 corresponding primers, each primer having a tail containing one or more tag sequences. Each combination of the tag sequences in the tail are different.

FIG. 3 shows another example of 8 target sequences and 8 corresponding primers, each primer having a tail containing one or more tag sequences. Each combination of the tag sequences in the tail are different from the combination of tag sequences in the tail of FIG. 2.

FIG. 4 shows an example of a schematic of the complex comprising genomic target sequence from the patient, a primer 1 with a synthetic variable region and a region corresponding to the genomic target sequence from the patient, and a primer 2 without the synthetic variable region.

FIG. 5 shows an example of a schematic of the complex comprising genomic target sequence from the patient, a primer with a synthetic variable region. Primer 1 and primer 2 have different combinations of tag sequences.

FIG. 6 shows an example of eight target sequences and 8 corresponding primers, each primer having a tail containing one or more tag sequences. Each combination of the tag sequences in the tail are different from each other. A combination of detection probes binds to the corresponding primer. Each detection probe is conjugated to a quencher and a fluorophore having an emission intensity level of 1 or 2. The schematic shows that each primer emits a unique combination of color and intensity of fluorophores.

FIG. 7 shows genomic DNA was isolated from a NSCLC FFPE sample and subjected to targeted massively parallel sequencing to identify tumor-specific variants. These variants were then used to design and assemble a tumor-specific dPCR detection assay sensitive for 8 different variants, as well as a reference target in EGFR. The FFPE-derived genomic DNA was then titrated into wild-type genomic DNA previously tested and confirmed not to contain these variants. The digital PCR detection assay was run in triplicate at a series of titration values.

FIG. 8 shows assay performance for the experiment in FIG. 7.

FIG. 9 shows plasma and matched CRC tumor sequencing data was obtained from three human cancer patients (A, B, and C), consents were collected, and experiments were carried out in accordance with industry standards and clinical protocols. Additional plasma samples from healthy human subjects were obtained for comparison. Cell free DNA (cfDNA) was isolated from all plasma samples using commercially available isolation kits. Tumor-specific dPCR assays based on each patient's tumor sequencing profile were designed and assembled to measure the “tracking variants” and run on isolated cfDNA from each plasma sample. In parallel, a targeted sequencing assay (“NGS”) was run on the same cfDNA samples. A wild-type “reference” target was measured in parallel as a positive control.

FIG. 10 shows three cancer cell lines (HCC1395, HCC1143, and HCC1187) were combined and used to inform the construction of a 26-target digital PCR assay. The cancer cell line mixture was titrated in a background of wild type genomic DNA (NA12878). All 26 targets were detected simultaneously in a single reaction at a 12.5% dilution, where each target had a unique tag sequence. The mean number of copies for each target is described in the “average copy” column (3 replicates).

FIG. 11 shows a two-fold dilution series of the cancer cell line mixtures from FIG. 10. The Y-axis reflects the cumulative copy number detected for all 26 targets. The assay illustrates linear sensitivity with an r²=0.998.

### DETAILED DESCRIPTION

### Interpretations and Definitions

Unless otherwise indicated, this description employs conventional chemical, biochemical, biotechnology, clinical biotechnology, molecular biology, immunology, cancer biology, clinical medicine, and pharmacology methods and terms that have their ordinary meaning to persons of skill in this field (unless otherwise defined/described herein). All publications, references, patents, and patent applications cited herein are hereby incorporated by reference in their entireties.

As used in this specification and the appended claims, the following general rules apply. Singular forms “a,” “an” and “the” include plural references unless the content clearly indicates otherwise.

As used herein, the following terms shall have the specified meaning. The term “about” takes on its plain and ordinary meaning of “approximately” as a person of skill in the art would understand, and unless specified otherwise, means plus or minus 10% of a value. The term “comprise,” “comprising,” “contain,” “containing,” “include,” “including,” “include but not limited to,” or “characterized by” is inclusive or open-ended and does not exclude additional, unrecited elements.

### Nucleic Acid Targets/Nucleic Acid Analytes

A nucleic acid target, also referred to as a nucleic acid analyte of the present disclosure may be derived from a sample. A biological sample may be a sample derived from a subject. A sample may comprise any number of macromolecules, for example, cellular macromolecules. A sample may comprise a plurality of cells. A sample may be a tissue sample, such as a biopsy, core biopsy, needle aspirate, and/or fine needle aspirate. The sample may be a tumor sample, including a solid tumor sample. A sample may be a fluid sample, including a blood sample, plasma sample, urine sample, or saliva sample. A sample may be a skin sample. A biological sample may be a cheek swab. A sample may be a plasma or serum sample. A sample may comprise one or more cells. The one or more cells may be derived from a tumor. A biological sample may be, for example, blood, plasma, serum, urine, saliva, mucosal excretions, sputum, stool, or tears. The sample may be obtained or derived from an environmental sample. For example, the sample may be a water sample or soil sample, or other samples found outside of a subject's body. The sample may be a wastewater sample. The sample may be a collection of samples. For example, a sample may be pooled with other sample and then subjected to methods described elsewhere herein.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. The nucleic acids may comprise a human genomic sequence. The nucleic acids may comprise an animal genomic sequence. The nucleic acids may comprise a plant genomic sequence. The nucleic acids may comprise a fungal genomic sequence. The nucleic acids may comprise an archaeal genomic sequence. The nucleic acids may comprise a pathogen associated sequence. The nucleic acid may comprise a wild type of sequence. The nucleic acid may comprise a variant sequence.

In certain embodiments, the nucleic acid target correlates with a lesion, benign tumor, pre-malignant tumor, malignant tumor, neoplasia, dysplasia, hyperplasia, hamartoma, and/or other pre-cancerous and cancerous conditions with abnormal cell growth in a patient. In some embodiments, the nucleic acid target encodes a patient-specific tumor variant sequence. For example, the patient-specific tumor variant sequence is encoded by oligonucleotides derived from the patient. As another example, the patient-specific tumor variant sequence may be determined by sequencing patient derived oligonucleotides, such as a DNA, including DNA derived from a buffy coat, tumor, and/or leukocytes.

In certain embodiments, the nucleic acid target correlates with or is associated with a relapse of a cancer. In certain embodiments, the cancer is a solid tumor. In certain embodiments, the cancer is a hematologic malignancy.

In certain embodiments, the nucleic acid target correlates with or is associated with a Minimal Residual Disease (MRD). In certain embodiments, the MRD may be a breast cancer; colorectal cancer; lung cancer, including non-small cell lung cancer (NSCLC) and/or small cell lung cancer (SCLC); melanoma; bladder cancer; ovarian cancer; gastric cancer; prostate cancer; pancreatic cancer; esophageal cancer; head and neck cancer; glioblastoma; sarcoma; thyroid cancer; renal cell carcinoma; hepatocellular carcinoma; cervical cancer; endometrial cancer; testicular cancer; neuroblastoma, and/or combinations thereof. In certain embodiments, MRD is a leukemia, preferably acute lymphoblastic leukemia (ALL), chronic lymphocytic leukemia (CLL), chronic myeloid leukemia (CML), and/or acute myeloid leukemia (AML); lymphoma, preferably non-Hodgkin's lymphoma (NHL), Hodgkin's lymphoma; follicular lymphoma, mantle cell lymphoma, T-cell lymphomas, precursor B-cell lymphoblastic lymphoma, diffuse large B-cell lymphoma (DLBCL), and/or Burkitt lymphoma; Waldenström's macroglobulinemia, multiple myeloma, myelodysplastic syndromes (MDS), and/or combinations thereof.

In certain embodiments the nucleic acid target may be associated with trisomy or fetal abnormalities. In certain embodiments, the nucleic acid target is derived from a virus. For example, the virus may comprise an influenza virus, coronavirus, respiratory syncytial virus, hepatitis virus, herpesvirus, papillomavirus, and/or combinations thereof.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, 100000, 500000, 1000000, 5000000, or 10000000 nucleotides, or more. In some instances, a member may be a gene. A nucleic acid target may comprise a gene whose detection may be useful in diagnosing one or more diseases and/or conditions. The gene may comprise a patient-specific tumor variant sequence. The gene may also comprise one or more single nucleotide polymorphisms (SNPs) or single nucleotide variants (SNVs). The disease and/or conditions may be cancer, a tumor, a relapse of cancer, an MRD, or combinations thereof.

A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses, bacteria, fungi) in a subject. The nucleic acid targets may be a human gene. The nucleic acid targets may be associated with a disease, such as cancer. The nucleic acid target may be a nucleic acid derived from an infectious agent. For example, the nucleic acid target may comprise a sequence of an influenza gene. The nucleic acid target may allow a genotype to be determined. The nucleic acid target may be a region of the human genome that indicates a predisposition for a particular disease.

For example, a particular mutation or SNP of in a subject may be associated with an increased risk of cancer relapse in a patient. For example, the detection of an increase in patient-specific tumor variant sequence over time may indicate the patient subject is at an elevated risk of cancer relapse and/or having MRD. As another example, the detection of a level of patient-specific tumor variant sequence above a threshold level may indicate the patient subject is at an elevated risk of cancer relapse and/or having MRD.

Nucleic acid targets may be of various concentrations in the reaction. The nucleic acid sample may be diluted or concentrated to achieve different concentrations of nucleic acids. The concentration of the nucleic acids in the nucleic acid sample may at least 1 genome copy equivalent per reaction, 2 genome copies equivalent per reaction, 5 genome copies equivalent per reaction, 10 genome copies equivalent per reaction, 20 genome copies equivalent per reaction, 30 genome copies equivalent per reaction, 40 genome copies equivalent per reaction, 50 genome copies equivalent per reaction, 100 genome copies equivalent per reaction, or more. In some cases, the concentration of the nucleic acids in the nucleic acid sample may be at most 0.1 genome copies equivalent per reaction, 0.2 genome copies equivalent per reaction, 0.5 genome copies equivalent per reaction, 1 genome copies equivalent per reaction, 2 genome copies equivalent per reaction, 3 genome copies equivalent per reaction, 5 genome copies equivalent per reaction, 10 genome copies equivalent per reaction, 20 genome copies equivalent per reaction, 40 genome copies equivalent per reaction, 50 genome copies equivalent per reaction, 100 genome copies equivalent per reaction, 1000 genome copies equivalent per reaction, 3000 genome copies equivalent per reaction, 5000 genome copies equivalent per reaction, 10000 genome copies equivalent per reaction or less.

The nucleic analytes may comprise mutations, such as single nucleotides variations, and the methods of the disclosure may be able to distinguish between analytes that differ by one or more nucleotides. For example, a first analyte may generate a first set of signals and a second analyte may generate a second set of signals, wherein the first analyte and second analyte differ by one nucleotide. The ability to distinguish two analytes may be based at least on the sequences of the oligonucleotides. For example, the oligonucleotides may be specific to a single nucleotide variant. For a molecular inversion probe-like oligonucleotide, the sequence at the end of the oligonucleotide may be single nucleotide specific or may be adjacent to the single nucleotide variant and detect the presence of the addition of a specific base. The ability to distinguish two analytes may be based on the presence of blocking groups. For example, a blocking group may be present that can be cleaved by an enzyme when a perfect duplex is formed and unable to be cleaved when a mismatch is present.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase refers to an enzyme that is capable of incorporating nucleotides to a strand of DNA in a template bound fashion. A polymerase may be Taq polymerase or a variant thereof. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exonuclease activity and degrade a probe resulting in a detectable signal. A nucleic acid enzyme may be capable, under appropriate conditions, of releasing a quencher from an oligonucleotide probe.

### Primers/Oligonucleotide Primers

In various aspects disclosed elsewhere herein, “primers” are used. Samples, mixtures, kits, and compositions of the present disclosure may comprise a primer, also referenced herein as an “oligonucleotide primer” or “amplification primer.” A primer of the present disclosure may be a deoxyribonucleic acid. A primer may be a ribonucleic acid. A primer may comprise one or more non-natural nucleotides. A non-natural nucleotide may be, for example, deoxyinosine. The oligonucleotide primer may be able to hybridize to a first analyte and a second analyte and may generates a first signal corresponding to said first analyte and a second signal corresponding to said second analytes.

A primer may comprise a first region complementary to an analyte and a second region comprising probe binding sites.

The analyte may be a patient derived oligonucleotide. The analyte may be a patient derived DNA. The analyte may be a patient derived RNA. The patient derived oligonucleotide may encode a patient-specific tumor variant sequence described elsewhere herein.

The second region may comprise one or more than one probe binding sites. Each probe binding site encode a unique tag sequence. In certain embodiments, the number of unique probe binding sites in a primer is one or more than one. In certain embodiments, the number of unique probe binding sites in a primer is two or more than two. In certain embodiments, the number of unique probe binding sites in a primer is three or more than three. In certain embodiments, the number of unique probe binding sites in a primer is four or more than four. In certain embodiments the number of unique probe binding sites in a primer is between one to npbs. In certain embodiments, n_{pbs} is two or more than two. In certain embodiments, n_{pbs} is one, or 2, or 3, 4, 5, 6, 7, 8, 9, 10, 11, or 12. The probe binding sites may be the same or different compared to other primers in a reaction mixture. The primer may comprise combinations of probe binding sites that are different than the probe binding sites of other primers in a reaction mixture.

In certain embodiments, the number of unique tag sequence in a primer is one or more than one. In certain embodiments, the number of unique tag sequences in a primer is two or more than two. In certain embodiments, the number of unique tag sequences in a primer is three or more than three. In certain embodiments, the number of unique tag sequences in a primer is four or more than four. In certain embodiments the number of unique tag sequences in a primer is between one to nₜₛ. In certain embodiments, nₜₛ is two or more than two. In certain embodiments, nₜₛ is one, or 2, or 3, 4, 5, 6, 7, 8, 9, 10, 11, or 12. The probe binding sites may be the same or different compared to other primers in a reaction mixture. The primer may comprise combinations of probe binding sites that are different than the probe binding sites of other primers in a reaction mixture.

The primers may comprise additional regions that another primer can anneal to. For example, a primer can comprise a universal region that a universal primer can anneal to. A first reaction using a primer comprising a universal region can anneal to a target and generate, via extension or amplification, a nucleic acid that comprise the target nucleic acid sequence and the universal region. In a second reaction, the universal primer may anneal to the universal region and generate additional copies of the nucleic acid. This may be especially advantageous for multiplexed workflows comprising multiple targets. For example, a mixture of different target specific primers that comprise universal regions can be used to amplify the multiple targets. As the resulting nucleic acids comprise universal regions, a universal primer can be used to amplify the multiple targets in a single reaction mixture, regardless of the original sequences of the targets.

As described elsewhere herein, amplification of targets can generate a signal via the degradation or removal of probes. Primers may comprise probe sites that may allow for targets to be labeled with probes sites via extension or amplification of the target specific primers. The probes may be allowed to anneal to the probe sites, and a second extension or amplification reaction may be performed to displace or degrade the probes, thereby generating a signal. This may be used in conjunction with primers with universal regions and the probe sites (and probes) may be three' to the universal region. The universal primers can then be used to generate the probe signal and can allow for multiplexed generation of signals from multiple targets.

A primer may comprise filler sequences. For example, a primer may comprise a sequence that does not anneal to a target, probe, or another primer. The filler sequence may have low or no binding to other sequences in the mixture. The filler sequence may be used to generate different primers that have a same or similar length that perform different functions. For example, a first primer may comprise two different probe binding sites and second primer may comprise one probe binding site and a filler sequence. The first primer may be able to bind two probes and generate two different signals, whereas the second primer may anneal to only one probe a generate one signal. Using the filler sequences the primers may be of comparable size may allow for improved multiplexing, for example, due to more similar melting temperatures or suitable reaction temperatures for the two primers.

A primer may comprise a blocking group or blocking region. The blocking group be at a three' end of an oligonucleotide. A blocking group may be unextendible and may need to be cleaved to allow a primer to be extended. The blocking group may allow for primers to differentiate between different loci or alleles, for example, those with single nucleotide polymorphisms (SNPs). For example, a blocking group may be unextendible and may need to be cleaved by an enzyme. The enzyme may recognize a perfectly matched primer-target duplex and may cleave the blocking group allowing for extension. A mismatched primer-target duplex may be unable to be recognized by the enzyme and fail to cleave off the blocking group, thereby blocking extension.

A primer may be a forward primer. A primer may be a reverse primer. In certain embodiments, the length of a primer may be between about five and about 150 nucleotides. In certain embodiments, the length of a primer may be at least 5, 10, 11, 12, 13, 14, 15, 16, 17, 18, 19, 20, 25, 30, 35, 40, 45, 50, 100, or 150 base pairs in length, or more. In certain embodiments, the length of a primer may be at most 150, 100, 50, 45, 40, 35, 30, 25, 20, 19, 18, 17, 16, 15, 14, 13, 12, 11, 10, or 5 nucleotides in length. In certain embodiments, the length of a primer may be about 5, 10, 11, 12, 13, 14, 15, 16, 17, 18, 19, 20, 25, 30, 35, 40, 45, 50, 75, 100, or 150 base pairs in length.

A set of primers may comprise paired primers. Paired primers may comprise a forward primer and a reverse primer. A forward primer may be configured to hybridize to a first region (e.g., a 3′ end) of a nucleic acid sequence, and a reverse primer may be configured to hybridize to a second region (e.g., a 5′ end) of the nucleic acid sequence, thereby being configured to amplify the nucleic acid sequence under conditions sufficient for nucleic acid amplification. Different sets of primers may be configured to amplify different nucleic acid target sequences. For example, a first set of primers may be configured to amplify a first nucleic acid sequence of a given length, and a second set of primers may be configured to amplify a second nucleic acid sequence of shorter length than the first nucleic acid sequence. In another example, a first set of primers may be configured to amplify a first nucleic acid sequence of a given length, and a second set of primers may be configured to amplify a second nucleic acid sequence of longer length than the first nucleic acid sequence.

A mixture may comprise a plurality of forward primers and/or reverse primers. A plurality of forward primers and/or reverse primers may be a deoxyribonucleic acid. Alternatively, a plurality of forward primers and/or reverse primers may be a ribonucleic acid. A plurality of forward and/or reverse primers may be between about five and about 50 nucleotides in length. A plurality of forward and/or reverse primer may be at least 5, 10, 11, 12, 13, 14, 15, 16, 17, 18, 19, 20, 25, 30, 35, 40, 45, or 50 base pairs in length, or more. A plurality of forward primer and/or reverse may be at most 50, 45, 40, 35, 30, 25, 20, 19, 18, 17, 16, 15, 14, 13, 12, 11, 10, or 5 nucleotides in length.

A set of primers (e.g., a forward primer and a reverse primer) may be configured to amplify a nucleic acid sequence of a given length (e.g., may hybridize to regions of a nucleic acid sequence a given distance apart). The nucleic acid sequence may be encoded in a nucleic acid target. Aspects of nucleic acid sequence and nucleic acid target are disclosed elsewhere herein. A pair of primers may be configured to amplify a nucleic acid sequence of a length of at least 20, at least 30, at least 40, at least 50, at least 75, at least 100, at least 125, at least 150, at least 175, at least 200, at least 225, at least 250, at least 275, or at least 300 base pairs (bp), or more. A pair of primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, the primer may be configured to hybridize, anneal or be homologous to sequences derived from humans. The sequence may be a sequence associated with cancer. The sequence may be associated with trisomy or fetal abnormalities. In some aspects, a mixture may include one or more synthetic (or otherwise generated to be different from the target of interest) primers for PCR reactions.

In some aspects, a mixture may be subjected to conditions sufficient to anneal a primer to a nucleic acid molecule. In some aspects, a mixture may be subjected to conditions sufficient to anneal a plurality primer to a nucleic acid molecule.

In some aspects, a mixture may be subjected to conditions sufficient to anneal a plurality of primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that a primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. A primer pair may be degraded by the RNase activity of a nucleic acid enzyme. Degradation of the primer pair may result in release of the primer. Once released, the primer pair may bind or anneal to a template nucleic acid molecule.

### Probes/Detection Probes/Oligonucleotide Probes

In various aspects disclosed elsewhere herein, “probes” are used. Samples, mixtures, kits, and compositions of the present disclosure may comprise a probe, also referenced herein as a “detection probe” or “oligonucleotide probe.” A probe may be a nucleic acid (e.g., DNA, RNA, etc.). A probe may comprise a region complementary to a region of a nucleic acid target. The nucleic acid target may be a nucleic acid encoding a unique patient-specific tumor variant sequence. The concentration of a probe may be such that it is in excess relative to other components in a sample.

The probe may be able to hybridize to one or more corresponding analyte such that each probe-analyte complex would generate a corresponding signal.

A probe may comprise a non-target-hybridizing sequence. A non-target-hybridizing sequence may be a sequence which is not complementary to any region of a nucleic acid target sequence. A probe comprising a non-target-hybridizing sequence may be a hairpin detection probe. A probe comprising a non-target hybridizing sequence may be a molecular beacon probe. A probe comprising a non-target hybridizing sequence may be a molecular inversion probe. Examples of molecular beacon probes are provided in, for example, U.S. Pat. No. 7,671,184, incorporated herein by reference in its entirety. An probe comprising a non-target-hybridizing sequence may be a molecular torch. Examples of molecular torches are provided in, for example, U.S. Pat. No. 6,534,274, incorporated herein by reference in its entirety.

A sample may comprise more than one probe. Multiple probes may be the same or may be different. A probe may be at least Iₚ nucleotides in length or at most Iₚ in length. In some embodiments, Iₚ can be 5 10, 11, 12, 13, 14, 15, 16, 17, 18, 19, 20, or more. In other embodiments, Iₚ can be 2, 3, 4, 5, 6, 7, 8, 9, 10, 20, 30, 40, or 50. In some examples, a mixture comprises No unique probes. In some cases, Nₚ can be 2, 3, 4, 5, 6, 7, 8, 9, 10, 11, 12, 13, 14, 15, 20, 40, 60 or more. In other cases, Nₚ can be 4, 8, 12, 24, 36, 48, 60.

As will be recognized and is described elsewhere herein, a probe may comprise a signal tag or a plurality of unique signal tags, which are described in more detail in other sections of this Application.

A probe may correspond to a region of a nucleic acid target. For example, a probe may have complementarity and/or homology to a region of a nucleic acid target. A probe may comprise a region which is complementary or homologous to a region of a nucleic acid target. In certain embodiments, a probe may have greater than 95% complementarity to a sequence of oligonucleotides on a nucleic acid target among a plurality of unique nucleic acid targets. In some embodiments, a probe may have less than 50%, 40%, 30%, 20%, 10%, 5%, or 1% complementarity to any member of a plurality of nucleic acid targets. A probe may have no complementarity to any member of the plurality of nucleic acid targets.

A probe corresponding to a region of a nucleic acid target may be capable of binding to the region of the nucleic acid target under appropriate conditions (e.g., temperature conditions, buffer conditions, etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be a molecular inversion probe or comprise a structure similar to a molecular inversion probe. For example, a probe may comprise (i) a first region at a first end of the probe that anneals to a nucleic acid target and (ii) a second region at a second end of the probe that anneals to the nucleic acid target at a different sequence. The oligonucleotide probe, when annealed to the target, may be able to be circularized via additional reactions, and generate a circularized probe. The oligonucleotide may be able to anneal to other probes (e.g., Taqman probes) and may comprise one or more probe binding sites. For example, an oligonucleotide may comprise from 5′ to 3′ (i) first region complementary to an analyte and a second region comprising probe binding sites, and a third region complementary to the analyte at different sequence. The second region may comprise more than one probe binding sites. The second region may comprise more than two probe binding sites. The second region may comprise more than three probe binding sites. The second region may comprise more four probe binding sites. The probe binding sites may be the same or different compared to other oligonucleotides in a reaction mixture. The oligonucleotide may comprise combinations of probe binding sites that are different than the probe binding sites of other oligonucleotides in a reaction mixture.

A probe may comprise filler sequences. For example, a probe may comprise a sequence that does not anneal to a target, primer, or another probe. The filler sequence may have low or no binding to other sequences in the mixture. The filler sequence may be used to generate different probes that have a same or similar length that perform different functions. For example, a first probe may comprise two different probe binding sites and second probe may comprise one probe binding site and a filler sequence. The first probe may be able to bind two probes and generate two different signals, whereas the second primer may anneal to only one probe a generate one signal. Using the filler sequences the probe may be of similar size may allow for improved multiplexing, for example, due to more similar melting temperatures or suitable reaction temperatures for the two probes.

A probe may comprise an uracil or other base that can be selectively recognized by an enzyme. For example, probe may comprise an uracil and may be cleaved via recognition of a Uracil-DNA glycosylases (UDG). For example, the probe may be circularized and then subsequently cleaved by a UDG.

The probe may be a universal probe. The probe may be non-specific to a specific analyte and bind to a region that is present in multiple different nucleic acids. As described throughout the disclosure, nucleic acids may be generated that have probe binding sites, such as via extension of a tailed primer or circularization of molecular inversion probe. These probe binding site may be universal probe binding sites, such that the sequence is common across different nucleic acids. Thus, the addition of a universal probe molecule can allow for binding to multiple different molecules and generating a signal from multiple molecules. For example, a FAM probe may have a set universal sequence. For a nucleic acid to generate a FAM signal, a sequence that binds to the FAM probe may be a part of the tail of the primer. When encoding or barcoding analytes, the primers or probe may be designed to use the universal probe binding sequences to generate the signal associated with that probe.

In some aspects, the probe may be configured to hybridize, anneal or be homologous to a nucleic acid molecule, such as a “nucleic acid target” or “nucleic acid target.” In some aspects, the probe may be configured to hybridize, anneal or be homologous to sequences derived from humans. The sequence may be a sequence associated with cancer. In certain embodiments, the nucleic acid target may comprise a tumor variant sequence. A “tumor variant sequence” may correlate with a lesion, benign tumor, pre-malignant tumor, malignant tumor, neoplasia, dysplasia, hyperplasia, hamartoma, and/or other pre-cancerous and cancerous conditions with abnormal cell growth in a patient. In some embodiments, the tumor variant sequence is a patient-specific tumor variant sequence. For example, the patient-specific tumor variant sequence encoded by oligonucleotides derived from the patient. As another example, the patient-specific tumor variant sequence may be determined by sequencing patient derived oligonucleotides, such as a DNA, including DNA derived from a buffy coat, tumor, and/or leukocytes.

In certain embodiments, the tumor variant sequence correlates with or is associated with a relapse of a cancer. In certain embodiments, the cancer is a solid tumor. In certain embodiments, the cancer is a hematologic malignancy.

In certain embodiments, the tumor variant sequence correlates with or is associated with a Residual Disease (MRD). In certain embodiments, the MRD may be a breast cancer; colorectal cancer; lung cancer, including non-small cell lung cancer (NSCLC) and/or small cell lung cancer (SCLC); melanoma; bladder cancer; ovarian cancer; gastric cancer; prostate cancer; pancreatic cancer; esophageal cancer; head and neck cancer; glioblastoma; sarcoma; thyroid cancer; renal cell carcinoma; hepatocellular carcinoma; cervical cancer; endometrial cancer; testicular cancer; neuroblastoma, and/or combinations thereof. In certain embodiments, MRD is a leukemia, preferably acute lymphoblastic leukemia (ALL), chronic lymphocytic leukemia (CLL), chronic myeloid leukemia (CML), and/or acute myeloid leukemia (AML); lymphoma, preferably non-Hodgkin's lymphoma (NHL), Hodgkin's lymphoma; follicular lymphoma, mantle cell lymphoma, T-cell lymphomas, precursor B-cell lymphoblastic lymphoma, diffuse large B-cell lymphoma (DLBCL), and/or Burkitt lymphoma; Waldenström's macroglobulinemia, multiple myeloma, myelodysplastic syndromes (MDS), and/or combinations thereof.

In certain embodiments the Nucleic Acid Target may be associated with trisomy or fetal abnormalities. In certain embodiments, the nucleic acid target is derived from a virus. For example, the virus may comprise an influenza virus, coronavirus, respiratory syncytial virus, hepatitis virus, herpesvirus, papillomavirus, and/or combinations thereof.

A probe may be provided at a concentration Cₚ. In some cases, a second nucleic acid probe can be provided at a concentration of at least about nCₚ. In some cases, a second nucleic acid probe can be provided at a concentration of at most about nCₚ. In certain embodiments, n can be 2, 3, 4, 5, 6, 7, or 8. In certain embodiments, n can be more than 8. Cₚ may be any concentration of a nucleic acid probe. In some cases, Cₚ is at least 1 nM, 5 nM, 10 nM, 20 nM, 30 nM, 40 nM, 50 nM, 100 nM, 150 nM, 200 nM, 250 nM, 300 nM, 350 nM, 400 nM, 450 nM, 500 nM, 1000 nM, or greater. In some cases, Cₚ is from about 1 nM to about 50 nM, from about 1 nM to about 30 nM, and/or from about 1nM to about 25 nM. In some cases, Cₚ is at most 1000 nM, 500 nM, 450 nM, 400 nM, 350 nM, 300 nM, 250 nM, 200 nM, 150 nM, 100nM, or 50 nM.

### Signal Tag

The signal tag also referred to herein as a “detectable agent” is capable of producing a fluorescent signal, electrochemical signal, chemiluminescent signal, and/or another quantifiable signal.

In some embodiments, the signal tag can comprise a “detectable label” can be a fluorescent label, such as a fluorophore, a fluorophore/quencher pair. A detectable label may be a chemiluminescent label. A “fluorophore” may be, for example, FAM, TET, HEX, TAMRA, ROX, JOE, Cy3, Cy5, Cy5.5, Cal Fluor Gold 540, Cal Fluor Orange 560, Cal Fluor Red 590, Cal Fluor Red 610, Cal Fluor Red 635, Quasar 570, Quasar 670, Quasar 705, or a derivative thereof, or an equivalent thereof. A fluorophore may be FAM. A fluorophore may be HEX. A quencher may inhibit signal generation from a fluorophore. A “quencher” may be, for example, TAMRA, BHQ-1, BHQ-2, BHQ-3, lowa Black, ZEN, Dabcy or an equivalent thereof. A quencher may be BHQ-1. A quencher may be BHQ-2. A quencher may be BHQ-3. In some embodiments, the signal tag can be a magnetic particle, and/or electrets structures exhibiting a permanent dipole.

Each probe used in the methods and assays of the presence disclosure may comprise at least one fluorophore. A fluorophore may be selected from any number of fluorophores. A set of fluorophores may be selected from 3, 4, 5, 6, 7, 8, 9, or 10 fluorophores, or more. One or more probes used in a single reaction may comprise the same fluorophore or the same set of fluorophores. In some cases, all probes used in a single reaction comprise the same fluorophore or the same set of fluorophores. Each probe may, when excited and contacted with its corresponding nucleic acid target, generate a signal. A signal may be a fluorescent signal. A plurality of signals may be generated from one or more probes.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In some cases, each probe in a mixture of a plurality of probes may comprise a same or similar signal tag. For example, a probe may comprise an identical signal tag to another probe. In some cases, a probe in a plurality of probes may comprise a different signal tag. In some cases, each probe comprises a different signal tag.

In some case, each fluorophore signal tag is capable of being detected in a single optical channel. In other case, a fluorophore may be detected in multiple channels. In some cases, a probe may have similar sequence or be capable or binding a similar sequence as another probe in the sample. In some cases, a probe may have a different sequence or be capable of binding a different sequence as compared to another probe in the sample.

### Signal Generation

Thermal cycling may be performed such that one or more probes are degraded by a nucleic acid enzyme. A probe may be degraded by the exonuclease activity of a nucleic acid enzyme. A probe may generate a signal upon degradation. In some cases, a probe may generate a signal only if at least one member of a plurality of nucleic acid targets is present in a mixture.

In various aspects, extension reactions and amplification reactions may be used to allow for the generation of signals. The extension reaction and amplification reaction may be used to generate a signal correspond to an analyte. The extension reaction may extend an oligonucleotide that can hybridize to more than one analyte. Based on the hybridization partner, the extension reaction may generate a different signal. Extension or amplification of a first analytes may generate a first signal whereas the extension or amplification of a second analyte may generate a second signal. The efficiency of the hybridization reactions may affect the extension reaction or the generation of a signal. The extension or amplification reaction may generate a signal by degrading or reaction with the oligonucleotide that can hybridize to more than one analyte. The oligonucleotide that can hybridize to more than one analyte may be a probe, and the extension or amplification reaction may allow generation of a signal from the probe. The probe may hybridize with different efficiency or affinity and may allow the generation of a different signal based on the analyte hybridized thereto.

Signal generation may correspond to reactions conditions of reactions relating to signal generation. The signal generation may be altered by a hybridization efficiency of the oligonucleotide. For example, an oligonucleotide may have a hybridization efficiency to a first analyte and a different hybridization efficiency to a second analyte which may in turn affect the generation of signal or alter the resulting signal that is generated. In the case of amplification or extension reactions, a time period or temperature may be altered such to change the signal generation efficiency or a kinetic signature shape. For example, a sample may comprise more than one analyte and an oligonucleotide that can hybridize to more than one analyte may be added to the sample. The different signal generation efficiency or kinetic shape of a reaction may be used to differentiate a first analyte and a second analyte. The annealing temperature of a reaction may be altered such that the hybridization to one analyte is favored over the hybridization to another analyte. Multiple reactions may be performed at different annealing temperature (for example using a gradient) that allows for a signal to be generated and distinguishable for different analytes. The reactions may be performed such that a first reaction has a more stringent annealing condition compared to a second reaction. The reactions may comprise an annealing time, and the annealing time may be modulated to affect the generation of a signal. For example, a first reaction may comprise an annealing time that is longer than an annealing time for a second reaction. For example, a first reaction may comprise an annealing time that is shorter than an annealing time for a second reaction. Similarly, extension times and temperatures may be modulated to affect the generation of a signal and allow different signal to be obtained based on the analyte. For example, a first reaction may comprise an extension time that is longer than an extension time for a second reaction. For example, a first reaction may comprise an extension time that is shorter than an extension time for a second reaction. For example, a first reaction may comprise an extension temperature that is higher (or lower) than an extension temperature for a second reaction. For example, a first reaction may comprise an extension temperature that is lower than an extension temperature for a second reaction.

A reaction may generate one or more signals. A reaction may generate a cumulative intensity signal comprising a sum of multiple signals. A signal may be a chemiluminescent signal. A signal may be a fluorescent signal. A signal may be generated by a probe. For example, excitation of a probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

As will be recognized and is described elsewhere herein, a signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more probes. Multiple signals may be generated by a probe. For example, an oligonucleotide may be able to bind to multiple analytes and may generate a signal corresponding to hybridization with a first analyte and a second signal corresponding with a second analyte.

S_{c} may be a number of signals detected in a single optical channel in an assay of the present disclosure. S_{c} may be at least 2, 3, 4, 5, 6, 7, 8, 9, 10, 11, 12, 13, 14, 15, 20, 25, 30, 40, 50 or more. S_{c} may be at most 50, 40, 30, 24, 20, 15, 14, 13, 12, 11, 10, 9, 8, 7, 6, 5, 4, 3, or 2. S_{c} may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In certain embodiments, S_{All} may be the number of signals detected in all optical channel in an assay of the present disclosure. In certain embodiments, S_{All}=C×S_{c}, wherein C is the number of optical channels used to detect the signals. In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where a probe comprises a signal tag, the probe may be degraded when bound to a region of a primer, thereby generating a signal. For example, a probe (e.g., a TaqMan® probe) may generate a signal following hybridization of the probe to a nucleic acid and subsequent degradation by a polymerase (e.g., during amplification, such as PCR amplification). A probe may be degraded by the exonuclease activity of a nucleic acid enzyme.

As will be recognized and is described elsewhere herein, a probe may comprise a quencher and a fluorophore, such that the quencher is released upon degradation of a probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a “signal intensity threshold.” A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules. The presence of a plurality of signals may be correlated with a combination of targets. The presence of a plurality of signals may be correlated with a unique combination of targets. For example, the detection of a particular plurality of signals may indicate the presence or absence of a unique or particular combination of targets.

### Digital Assays

In some aspects, the present disclosure provides methods for performing a digital assay. A method for performing a digital assay may comprise partitioning a plurality of nucleic acid targets and a plurality of probes into a plurality of partitions. In some cases, two, three, four, five, or more nucleic acid targets may be partitioned into a plurality of partitions together with two, three, four, five, six, seven, eight, nine, ten, 11, 12, 13, 14, 15, 16, 17, 18, 19, 20 or more probes. Following partitioning, the nucleic acid targets may be amplified in the partitions, for example, by polymerase chain reaction (PCR). Next, S signals may be generated from the oligonucleotide probes. Each signal of the S signals may correspond to the presence of a unique combination of nucleic acid targets in a partition. Following signal generation, the S signals may be detected in a single optical channel. The signals may be detected using, for example, fluorescence detection in a single-color channel.

A method for performing a digital assay may comprise amplifying nucleic acid targets derived from a sample in a plurality of partitions comprising probes complementary to one or more regions of nucleic acid targets. Each probe may be labeled with a fluorophore. The fluorophores may be capable of being detected in a single optical channel. For example, the fluorophores may each comprise similar emission wavelength spectra, such that they can be detected in a single optical channel. Following partitioning, S signals may be detected from the plurality of partitions if one or more of the nucleic acid targets is present. Each of the S signals may correspond to a unique combination of one or more of the nucleic acid targets present in a partition. From the S signals, the presence or absence of each of the nucleic acid targets in the sample may be determined.

At least one signal of the plurality of signals may correspond with the presence of a unique combination of two or more of the first or second pluralities of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the first or second pluralities of nucleic acid molecules in a single partition (e.g., may be an ambiguous signal). For example, a signal may correspond with the presence of one nucleic acid molecule and may also correspond with the presence of two nucleic acid molecules.

A reaction may comprise generating a cumulative signal measurement. Assays of the present disclosure may comprise comparing two or more cumulative signal measurements to unambiguously detect any combination of nucleic acid targets in a sample. A cumulative signal measurement may comprise one or more signals generated from one or more probes provided to a sample solution. A cumulative signal measurement may be a signal intensity level which corresponds to the sum of signals generated from multiple oligonucleotide probes.

For example, two probes may each bind to different regions of the same nucleic acid molecule, where each probe generates a signal of a given wavelength at a Sₗ, the signal intensity level for the first probe binding to the nucleic acid molecule is Su and the signal intensity level for the first probe binding to the nucleic acid molecule is Sₗ₂. Measurement of these signals would generate a cumulative signal measurement corresponding to the sum of both signal intensities (Sₗ+Sₗ₂). In some cases, Sₗ₁ equals Sₗ₂, providing a cumulative signal measurement of 2Sₗ₁ or 2Sₗ₂. In some embodiments, Su is different from Sₗ₂. In certain embodiments, Sₗ₂=nSₗ₁, where n is a number ≥1. In certain embodiments, n is a number selected from the list 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In certain embodiments, n is a number selected from the list 1, 2, 4, 8, 16, 32, 64, 128, 256, 512, and 1024. In certain embodiments, P is the number of unique probes that has binding specificity to a unique region on the same nucleic acid molecule. In certain embodiments, P is a number selected from the list 1, 2, 3, 4, 5, 6, 7, 8, 9, 10, and 11. In certain embodiments, each of P probes will generate a S_{lP}. In certain embodiments, the Sₗ for each probe is selected from the list consisting of 2Sₗ₁=Sₗ₂, 4Sₗ₁=Sₗ₃, 8Sₗ₁=Sₗ₄, 16Sₗ₁=Sₗ₅, 32Sₗ₁=Sₗ₆, 64Sₗ₁=Sₗ₇, 128Sₗ₁=Sₗ₈, 256Sₗ₁=Sₗ₉, 512Sₗ₁=Sₗ₁₀, 1024Sₗ₁=Sₗ₁₁, and combinations thereof. In certain embodiments, the cumulative intensity S_{cum} is selected from the list of 1, 3, 7, 15, 31, 63, 127, 255, 511, 1023, and 2027.

As another example, two probes may each bind to a different nucleic acid molecule in a sample and/or a partition of a sample, where each probe generates a signal of a given wavelength at a Sₗ, and where the signal intensity level for the first probe binding to the nucleic acid molecule is Sₗ₁ and the signal intensity level for the first probe binding to the nucleic acid molecule is Sₗ₂. Measurement of these signals would generate a cumulative signal measurement corresponding to the sum of both signal intensities (Sₗ₁+Sₗ₂). In some cases, Sₗ₁ equals Sₗ₂, providing a cumulative signal measurement of 2Sₗ₁ or 2Sₗ₂. In some embodiments, Sₗ₁ is different from Sₗ₂. In certain embodiments, Sₗ₂=nSₗ₁, where n is a whole number ≥1. In certain embodiments, n is a number selected from the list 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In certain embodiments, n is a number selected from the list 1, 2, 4, 8, 16, 32, 64, 128, 256, 512, and 1024. In certain embodiments, P is the number of unique probes that is specific for an unique nucleic acid molecule in a sample. In certain embodiments, P is a number selected from the list 1, 2, 3, 4, 5, 6, 7, 8, 9, 10, and 11. In certain embodiments, each of P probes will generate a S_{lP}. In certain embodiments, the Sₗ for each probe is selected from the list of 2Sₗ₁=Sₗ₂, 4Sₗ₁=Sₗ₃, 8Sₗ₁=Sₗ₄, 16Sₗ₁=Sₗ₅, 32Sₗ₁=Sₗ₆, 64Sₗ₁=Sₗ₇, 128Sₗ₁=Sₗ₈, 256Sₗ₁=Sₗ₉, 512Sₗ₁=Sₗ₁₀, 1024Sₗ₁=Sₗ₁₁, and combinations thereof. In certain embodiments, the cumulative intensity S_{cum} is selected from the list consisting of 1, 3, 7, 15, 31, 63, 127, 255, 511, 1023, and 2027.

Methods of the present disclosure may comprise partitioning a sample or mixture into a plurality of partitions. A sample of mixture may comprise nucleic acids, oligonucleotide probes, and/or additional reagents into a plurality of partitions. A partition may be a droplet (e.g., a droplet in an emulsion). A partition may be a microdroplet. A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids. Each droplet of a plurality of droplets may generate a signal. A plurality of signals may comprise the signal(s) generated from each of a plurality of droplets comprising a subset of a sample.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample may undergo an extraction to extract molecules used in the assay. For example, the extraction may use a column to bind or interact with a molecule. For example, an RNA extraction kit may be used such as a Qiagen RNA mini kit to extract or isolate RNA. A sample may be diluted. A sample may be diluted at least at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:10000, 1:100000 or more. A sample may be diluted at no more than at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:10000, 1:100000, or less. The sample may be diluted in a buffer or a solution. For example, the sample may be diluted in Tris-Ethylenediaminetetraacetic acid (TE) buffer. The sample may be diluted with a solution comprising alcohol. The sample may be diluted with a solution comprising sodium acetate.

### Reactions

In various aspects disclosed elsewhere herein, reactions are performed. A reaction may comprise contacting nucleic acid targets with one or more probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

In some aspects, the methods may comprise circularization reactions. Molecular inversion probes, or oligonucleotides with similar structures may be used such that probes may present in a configuration, upon annealing to a target, which may allow for the two ends of the probes to be connected. For example, the two ends may be directly adjacent to one another, and a ligation reaction may join the two ends together to generate a circularized nucleic acid. The two ends may be more than one nucleotide away from each other and may be subjected to gap fill reactions, such as polymerization reactions, extension reactions, or other reactions that attach additional nucleotides to the ends of the oligonucleotides. The gap fill reactions may then be followed by a ligation reaction to circularize the nucleic acid.

In some aspects, the methods may comprise exonuclease, cleavage, or nucleic acid degradation reactions. An exonuclease may be used to selectively degrade certain nucleic acids. For example, probes may be added to a mixture and allowed to anneal to targets and the probes may be circularized. Probes that did not anneal to a target may remain linear. An exonuclease may be used to selectively remove the linear probes that did not bind to any target, leaving the probes that bound to targets intact. The remaining probes may be subjected to additional reactions such to generate signals associated with the target and allow for detection of targets. Similarly, enzymes may selectively cleave at a base type or sequence. For example, the probe may be circularized and subjected to an exonuclease reaction that degrades non-circularized oligonucleotides (e.g., probes that did not anneal to a target). The circularized probe may then be subjected to a cleavage reaction to linearize the circularized probe and allow for additional reaction to be performed on the probes. The cleavage reaction may use a restriction enzyme that recognizes specific sequences. A cleavage reaction may use an enzyme that recognizes a specific base, such as a uracil-DNA glycosylase, which cleaves at a uracil base.

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The oligonucleotide may be able to amplify more than one sequence analyte by hybridizing upstream or downstream of multiple different sequences. The amplification reaction may comprise the use of nucleotide tri-phosphate reagents. The nucleotide tri-phosphate reagents may comprise using deoxyribo-nucleotide triphosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes; or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Process for Unambiguously Resolving the Identity of Multiple Targets

Disclosed herein are methods to unambiguously resolving the identities of multiple targets in the same volume of sample at the end of a dPCR reaction. When ambiguity to the identity of the genetic target present the partition arises from the signals encoded using the universal signal encoding scheme, a method to unambiguously resolve the identities can be implemented. For example, if three of the targets are given signals 1000, 0100, and 1100, a partition with a final signal reading of 1100 can be ambiguous, because it can potentially represent five different scenarios of target combination: (a) 1100, (b) 1000+0100, (c) 1100+1000, (d) 1100+0100, or (e) 1100+1000+0100.

To calculate a final value for the number of copies of each target present in the original sample, targets can be analyzed to resolve the ambiguity. In an exemplary scheme comprising of 4-channel signal, the analytic process starts with assigning every partition a signal based on its amplitude in each color channel compared to calibrator intensities. This uniquely determines which universal probe sequences is amplified in each partition, however, may leave ambiguity between different amplified target combinations that could have contributed those universal probe sequences. Assign every partition with a signal of 0000 as “null” because it contains no amplified targets. Count every “null” to arrive at a “null count,” representing the total number of 0000 partitions.

For partitions with a signal corresponding to a target with a probe in only one color channel (e.g. 1000, 0100 etc, which are known to contain exactly that single target and no others), compute the ratio between the number of these single-channel partitions to the null count, to arrive at the number of copies of the corresponding single-channel targets present. For partitions with a signal corresponding to a target with a probe in exactly two-color channels (e.g., 1100 etc.), it can represent more than one different target combinations. Using the concentration of all possible single-channel component targets (in this case, 1000 and 0100) computed above and the knowledge of all possible target combinations that can produce a 1100partition, compute the number of copies of the 1100 target that must be present such that all of the various combinations with the 1000 and 0100 targets add up to the proper number of 1100 partitions. Repeat this step for all targets with 2-channel signals to resolve all ambiguities within the 2-channel signals.

Then, repeat the process above for all targets with 3-channel signals (e.g., 1110 etc) and all targets with 4-channel signals to resolve all ambiguities and compute how many copies of each target are present in the original sample.

The analysis can be applied to a five-channel signal, to unambiguously compute all null signals (00000) and unambiguously resolve all targets with one-channel signals (e.g. 10000 etc), two-channel signals (e.g. 11000 etc), three-channel signals (e.g. 11100 etc), four-channel signals (e.g. 11110 etc), and five-channel signals. This method can be expanded and applied to any scheme across any number of color channels.

### Accuracy, Specificity, and Sensitivity

The method of the disclosure may be able to correctly identify the presence of analyte in a sample and can be measured in terms of the accuracy of the assay, the sensitivity of the assay, the specificity of the assay.

Accuracy may be calculated as the total number of correctly classified samples divided by the total number of samples, e.g., in a test population. In some embodiments, the methods herein show an accuracy or rate of accuracy, R_{A}, for predicting the presence of an analyte. In certain embodiments, R_{A} is at least about 75% (e.g., R_{A} may be at least about: 75%, 80%, 85%, 90%, 95%, 97%, 99% or 100%). In certain embodiments, RA is at least about 50% (e.g., RA may be at least about: 55%, 60%, 65%, 70%, 75%, 80%, 85%, 90%, 95%, or more).

Specificity is a measure of the “true negatives” that are predicted by a test to be negative and may be calculated as the number of correctly identified normal samples divided by the total number of normal samples. In certain embodiments, the methods herein show a specificity or rate of specificity, R_{S}, for predicting the presence of an analyte. In certain embodiments, R_{S} is at least about 75% (e.g., R_{S} may be at least about: 75%, 80%, 85%, 90%, 95%, 97%, 99% or 100%). In certain embodiments, R_{S} is at least about 50% (e.g., R_{S} may be at least about: 55%, 60%, 65%, 70%, 75%, 80%, 85%, 90%, 95%, or more).

Sensitivity is a measure of the “true positives” that are predicted by a test to be positive and may be calculated as the number of correctly identified cancer samples divided by the total number of cancer samples. In certain embodiments, the methods herein show a specificity or rate of sensitivity, R_{N}, for predicting the presence of an analyte. In certain embodiments, R_{N} is at least about 75% (e.g., Rₛ may be at least about: 75%, 80%, 85%, 90%, 95%, 97%, 99% or 100%). In certain embodiments, R_{N} is at least about 50% (e.g., R_{S} may be at least about: 55%, 60%, 65%, 70%, 75%, 80%, 85%, 90%, 95%, or more).

### Kits

The present disclosure provides kits for sample collection. The kit may comprise a sample collection vessel or sample collection tube. The kit may comprise a sample collection tool or an object that can obtain a sample via the contact of cells or nucleic acids from the subject and transfer sample to a sample collection vessel or tube. The sample collection tool may comprise a swab.

The present disclosure also provides kits for performing assays or analysis of assay results. Kits may comprise one or more probes. Probes may be lyophilized. Different probes may be present at different concentrations in a kit. Probes may comprise a fluorophore and/or one or more quenchers.

Kits may comprise one or more sets of primers or probes as described herein. The kits may further comprise a set of primers comprising paired primers. Paired primers may comprise a forward primer and a reverse primer. A set of primers may be configured to amplify a nucleic acid sequence corresponding to particular nucleic acid target. For example, a forward primer may be configured to hybridize to a first region (e.g., a 3′ end) of a nucleic acid sequence, and a reverse primer may be configured to hybridize to a second region (e.g., a 5′ end) of the nucleic acid sequence, thereby being configured to amplify the nucleic acid sequence. Different sets of primers may be configured to amplify nucleic acid sequences. In one example, a first set of primers may be configured to amplify a first nucleic acid sequence, and a second set of primers may be configured to amplify a second nucleic acid sequence. Primers configured to amplify nucleic acid molecules may be used in performing the disclosed methods. In some cases, all of the primers in a kit are lyophilized.

As will be recognized and is described elsewhere herein, the nucleic acid target may comprise a tumor variant sequence. In certain embodiments, the tumor variant sequence correlates with or is associated with a Minimal Residual Disease (MRD).

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase Ill may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading a probe. A nucleic acid enzyme may be capable, under appropriate conditions, of releasing a quencher from a probe. Kits may comprise instructions for using any of the foregoing in the methods described herein.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution to detect, analyze, evaluate, screen for, prognose, diagnose, and/or monitor a condition and/or a disease. In certain embodiments, the condition or disease may be a lesion, benign tumor, pre-malignant tumor, malignant tumor, neoplasia, dysplasia, hyperplasia, hamartoma, and/or other pre-cancerous and cancerous conditions with abnormal cell growth. In certain embodiments, the condition or disease may be a cancer. In certain embodiments, the condition or disease is a cancer involving a solid tumor. In certain embodiments, the condition or disease is a cancer involving a hematologic malignancy. In certain embodiments, the condition or disease is a relapse of a cancer. In certain embodiments, the condition or disease is a Residual Disease (MRD). In certain embodiments, the condition or disease is a breast cancer; colorectal cancer; lung cancer, including non-small cell lung cancer [NSCLC] and/or small cell lung cancer [SCLC]; melanoma; bladder cancer; ovarian cancer; gastric cancer; prostate cancer; pancreatic cancer; esophageal cancer; head and neck cancer; glioblastoma; sarcoma; thyroid cancer; renal cell carcinoma; hepatocellular carcinoma; cervical cancer; endometrial cancer; testicular cancer; neuroblastoma, and/or combinations thereof. In certain embodiments, the condition or disease is a leukemia, preferably acute lymphoblastic leukemia (ALL), chronic lymphocytic leukemia (CLL), chronic myeloid leukemia (CML), and/or acute myeloid leukemia (AML); lymphoma, preferably non-Hodgkin's lymphoma (NHL), Hodgkin's lymphoma; follicular lymphoma, mantle cell lymphoma, T-cell lymphomas, precursor B-cell lymphoblastic lymphoma, diffuse large B-cell lymphoma (DLBCL), and/or Burkitt lymphoma; Waldenström's macroglobulinemia, multiple myeloma, myelodysplastic syndromes (MDS), and/or combinations thereof.

The results obtained from the assay may be sent to the subject directly. The subject, medical provider, or other institution may be able to access the remote server such review or analyze the results. For example, the results may then be transmitted to another institution/or medical professional for monitoring or for providing recommendations for the subject. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor’ and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries, and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine-readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, f, or example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of Uls include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

### Measurable Residual Disease (MRD)

A measurable residual disease (MRD) is when a small number of cancer cells remain in a patient's body during or after treatment. Since these cancer cells are generally not detectable by traditional imaging or hematological examinations, detection of MRD is important to monitor and assess risk of relapse. While diagnosing and monitoring MRD is pivotal in evaluating the effectiveness of a cancer treatment, predicting cancer relapse, and guiding subsequent therapeutic decisions, because the number of residual cells post treatment is low and the heterogeneity of cancer cell markers are high, detection and quantification of MRD have posed significant challenges to clinical oncology. In general, MRD detection has been shown to be important in the management of hematological malignancies, such as leukemia, lymphoma, and multiple myeloma, where it serves as a powerful predictor of relapse and survival outcomes. As described herein, the methods, composition, systems, and macromolecules, provides novel, accurate, sensitive, effective, and economical ways to detect, analyze, evaluate, screen for, prognose, diagnose, and/or monitor, MRD in a patient.

Current techniques for MRD assessment include flow cytometry, polymerase chain reaction (PCR), and next-generation sequencing (NGS), each with its own limitations on sensitivity, specificity, cost, and applicability across different types of cancer. The embodiments disclosed in this application provides a surprisingly sensitive and cost-effective way to periodically monitor MRD and cancer relapse in patients. The embodiments disclosed herein are patient-specific and the procedure are scalable in a clinic or hospital setting.

### Patient-Specific Cancer Testing

The advancement in personalized medicine allows for developments in patient-specific cancer testing, allowing for the tailored cancer detection, monitoring, and treatment described herein. Using patient-specific tumor variant sequence identified through sequencing methods such as exome sequencing and/or deep target sequencing, the embodiments described herein can provide fast, sensitive, and effective monitoring of cancer progression and/or relapse.

### EXAMPLES

### Example 1

### Designing Patient-Specific Digital PCR Mastermix(es)

A set of patient-specific digital PCR mastermix(es) bespoke for the patient are designed to detect key tumor-specific variants using genomic DNA extracted from (a) the patient's tumor biopsy that is suspected to contain cancer mutations and (b) patient's buffy coat that is thought to contain normal patient DNA.

### A. Identify Tumor-Specific Variants in the Patient

In general, a sample of tumor tissue biopsy (a), which contains the patient's cancerous genetic content and a sample of normal tissue or a buffy coat of a matched blood sample (b), which provides a baseline for the patient's normal, non-cancerous genetic content, are collected from a cancer patient or a patient suspected of having cancer. A genomic DNA is isolated from the tissue biopsy in (a). A genomic DNA from leukocytes or the buffy coat, or from matched normal tissue sample is isolated from the tissue biopsy in (b). The isolated genomic sequences are each sequenced by exome sequencing OR deep targeted sequencing, but not whole genome sequencing. A typical workflow includes the steps of (i) optionally shearing high molecular weight genomic DNA from the tumor/normal sample into shorter fragments; (ii) performing an end-repair and A-tailing step; (iii) ligating a set of synthetic adapters to the A-tailed genomic DNA; (iv) performing a hybrid capture step, whereby biotinylated RNA baits/probes complementary to the regions of interest (e.g. exome) are hybridized to the library; (v) pulling down of the hybridized exome library using streptavidin beads; (vi) washing beads/digesting RNA; and (vii) performing PCR on the enriched library using two primers targeting the synthetic adapters ligated in step (iii). Sequencing can involve the SureSelect Human All Exon (Agilent).

A broad first set of patient-specific tumor variants is identified by comparing the sequenced tumor tissue biopsy DNA and the sequenced buffy coat DNA. A narrower second set of patient-specific tumor variants is developed by comparing the broad first set with known tumor variants published in external databases such as COSMIC, ClinVar, gnomAD, among others. A prioritized third set of patient-specific tumor variants (8-700+) is developed by selecting the tumor-specific variants suitable for dPCR monitoring. The selection criteria include: (a) measured tumor variant allele frequency, (b) variant prevalence in the population, (c) sequence context, (d) suitability for PCR detection (particularly dPCR), and (e) PCR compatibility with other variants in the sample.

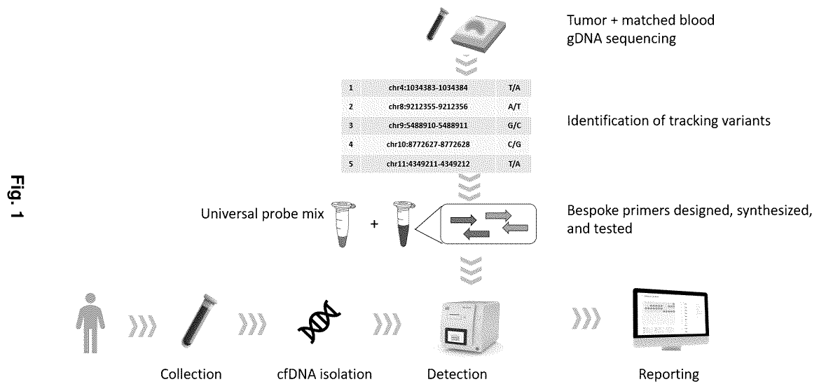

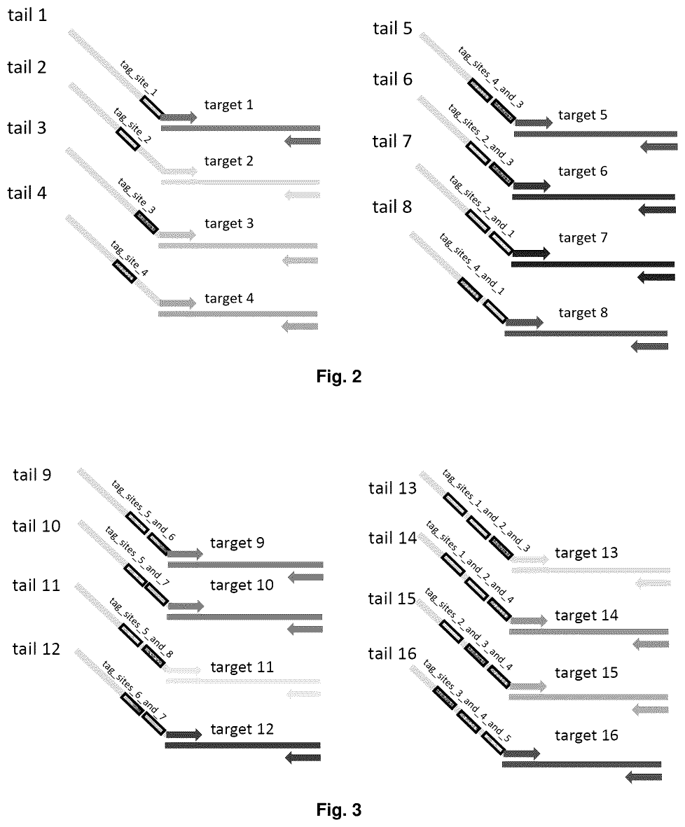

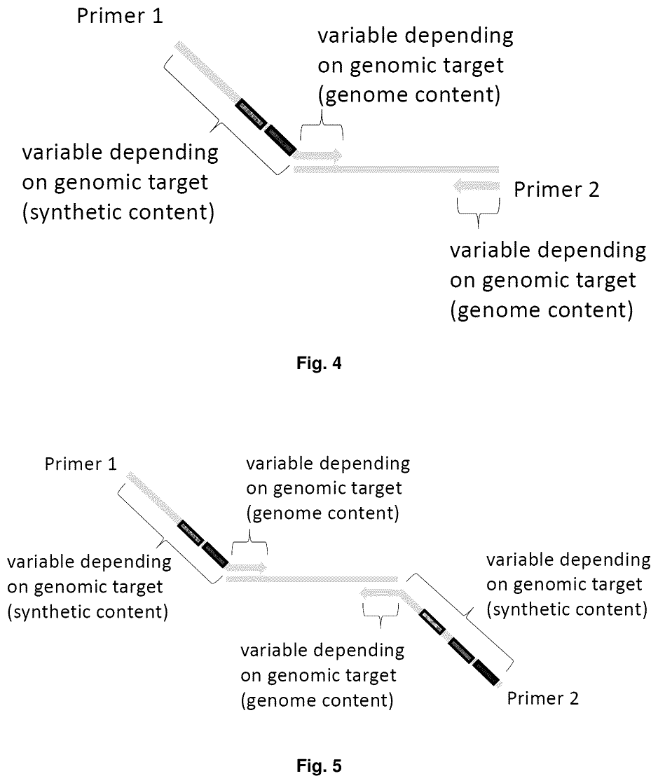

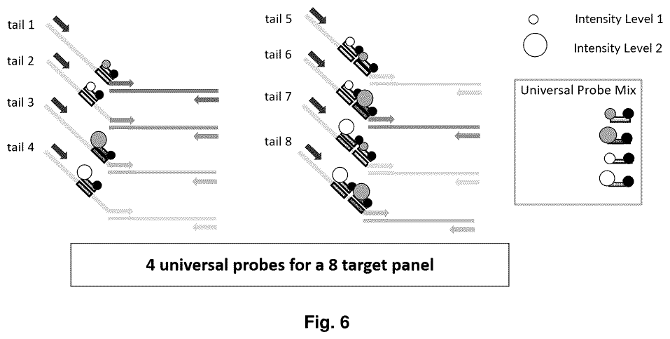

Candidate PCR primers for the prioritized third set of patient-specific tumor variants are designed and synthesized (see FIG. 1A and 1B, showing a set of 8 targets developed for subject A and subject B, respectively). Each of the 8 targets corresponds to the 8 sets of 16 unique primers for the 8 targets. The primer design corresponding to FIG. 2A is shown here, where only one primer from each set contains one or more synthetic variable region. However, the design from FIG. 2B can also be used.

The PCR detection systems optionally: (a) leverage tailed primers containing universal probe sequences; (b) leverage RNase H chemistry; (c) leverage patient-specific germline SNPs for sample tracking/quality control over time; (d) detect two-phased variants in close proximity (e.g. <500 nt) to improve specificity/reduce noise; and/or (e) detect strand-specific variants (e.g. detect both the variant on Watson strand and the Crick strand).

A candidate bespoke PCR detection system is created by (a) separating the PCR primers into one or more portions (i.e. 1-16 portions) and (b) combining the PCR primers with additional reagents to develop one or more sets patient-specific digital PCR mastermix(es), where the additional reagents include one or more of universal probe mix, DNA polymerase, RNase H, dNTPs, buffers, magnesium, among others, where one or more of the additional reagents are kept separate from the other (e.g. primer/probe vs enzyme components) until immediately prior to the reaction, where the annealing temperature is optionally less than the median melting temperature of the primers (e.g. 5-10° C. below), and where the bespoke PCR detection system is theoretically capable of detecting all of the tumor-specific variants in Step 6 (i.e. 8-1000+).

The performance of the candidate bespoke PCR detection systems is assessed using control templates and high throughput methods (e.g., using synthetic template mixtures and massively parallel sequencing and/or digital PCR as the readout). Based on the results of these assessments, the optimal performing PCR primers are selected for the finalized bespoke PCR detection system

### Assessing the Quality of the Primers

The specificity of the primers is tested. The specificity test includes: (A) pooling (i) up to 48 candidate primers in a PCR reaction and (ii) synthetic nucleic acid comprising target loci, OR (B) pooling (i) candidate primers and (ii) synthetic nucleic acid samples comprised of up to 48 normal target loci, and (C) sequencing the products. The specificity test shows amplification.

The sensitivity of the primers is tested. The sensitivity test includes (A) pooling (i) up to 48 candidate primers in a PCR reaction and (ii) synthetic nucleic acid comprising target loci, OR (B) pooling (i) candidate primers, and (ii) synthetic nucleic acid comprised of up to 48 patient-specific tumor variants, and (C) sequencing the products. The specificity test shows strong target detection and amplification.

The specificity of the primers can also be tested by (A) pooling (i) up to 48 candidate primers in a PCR reaction and (ii) synthetic nucleic acid comprising target loci, OR (B) pooling (i) candidate primers, and (ii) a combination of synthetic nucleic acid with (a) normal target loci, (b) patient-specific tumor variants, and (c) combinations thereof, and (C) sequencing the products. The specificity test shows strong target detection and amplification.

The specificity and/or sensitivity of the primers can also be tested by (A) pooling (i) up to 48 candidate primers in a PCR reaction and (ii) synthetic nucleic acid comprising target loci, OR (B) pooling (i) candidate primers, and (ii) a combination of synthetic nucleic acid comprised of (a) normal target loci, (b) patient-specific tumor variants, and (c) combinations thereof, wherein the total number of unique (a), (b) and (c) does not exceed 48, and (C) sequencing the products. The specificity test shows strong target detection and amplification.

The specificity and/or sensitivity of the primers can also be tested by (A) pooling (i) up to 48 candidate primers in a PCR reaction and (ii) synthetic nucleic acid comprising target loci, OR (B) pooling (i) candidate primers, and (ii) a combination of synthetic nucleic acid comprised of (a) normal target loci, (b) patient-specific tumor variants, (c) other tumor-specific variants, and (d) combinations thereof, wherein the total number of unique (a), (b), (c) and (d) does not exceed 48, and (C) sequencing the products. The specificity test shows strong target detection and amplification.

### Example 2

### Monitoring the Patent's Cancer Progression

Samples of cell-free DNA is periodically collected from the patient's plasma. Digital PCR is performed using the patient-specific master PCR mixes. Tumor-specific variants are monitored based on the individually unique primer tags designed-in the patent specific digital PCR mastermix(es) in Example 1

### A. Periodic Collection of Patient's Cell-Free DNA

Blood sample from cancer patients are collected periodically to isolate (a) plasma and (b) leukocytes/buffy coat (control). Cell-free DNA are then isolated from the plasma and genomic DNA are isolated from leukocytes or buffy coat. The cell-free DNA and genomic DNA are tested for quality and quantity. Genomic regions containing variants of interest for the patient are set up in a pre-amplified reaction. The pre-amplified materials are split between 16 multiple parallel reactions. Digital PCRs are performed using the bespoke patient-specific digital PCR mastermixes to measure tumor-specific variants on the cell-free DNA and genomic DNA.

The results of the digital PCR data are analyzed automatically in a software on the cloud to determine (a) identity of the variants present, (b) copy numbers of the identified variants, and the (c) allele frequency of the identified variants. A report is generated to provide (a) individual variant information, (b) assessment of overall tumor load in circulation, and (c) circulating tumor load trajectory information based on the longitudinal series of timepoints.

### Example 3

### NSCLC FFPE gDNA in cfDNA Background, 8 Targets

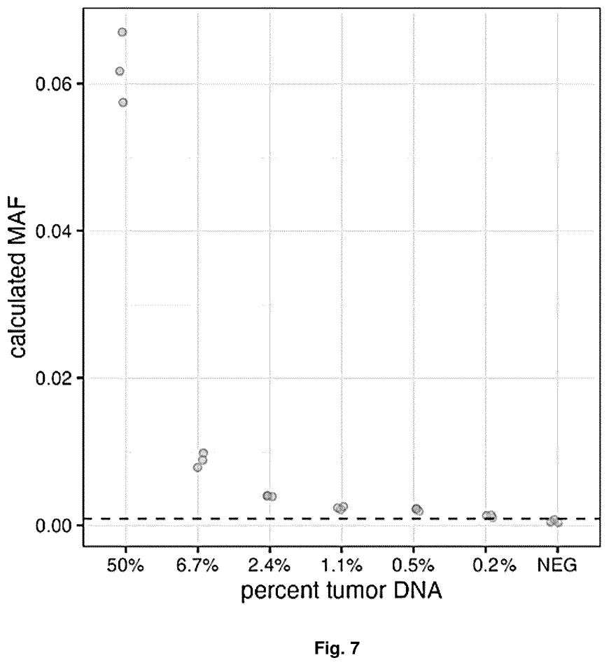

Genomic DNA are isolated from a NSCLC FFPE sample and are subjected to targeted massively parallel sequencing to identify tumor-specific variants. These variants are then used to design and assemble a tumor-specific dPCR detection assay sensitive for 8 different variants, as well as a reference target in EGFR. The FFPE-derived genomic DNA is then titrated into wild type genomic DNA previously tested and confirmed not to contain these variants. The digital PCR detection assay is run in triplicate at a series of titration values. The results are shown in FIG. 7. Dashed line indicates threshold used to distinguish negative samples from samples with a postive spike in. Negative samples are indicated in red. Positive samples are indicated in green.

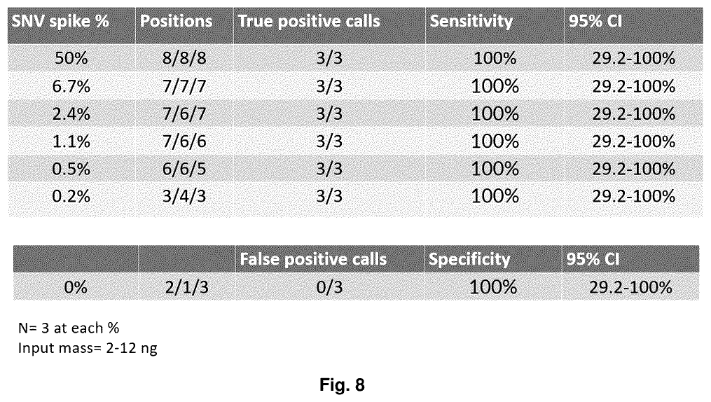

The assay performance for the experiment shown in FIG. 7 is shown in FIG. 8. The position column shows the number of targets detected within the each of the replicates. As the variant levels decrease, the number of targets detected is reduced as all are not present in equal amounts in the NSCLC FFPE sample. All assays are do not have 100% specificity. As a result some targets are detected at low level copies within the negative sample (0%) but the cumulative copy detection at 0.2% is still greater and are able to be distinguished using a single threshold for all samples.

### Example 4

### Tumor Informed Bespoke dPCR on Matched Plasma

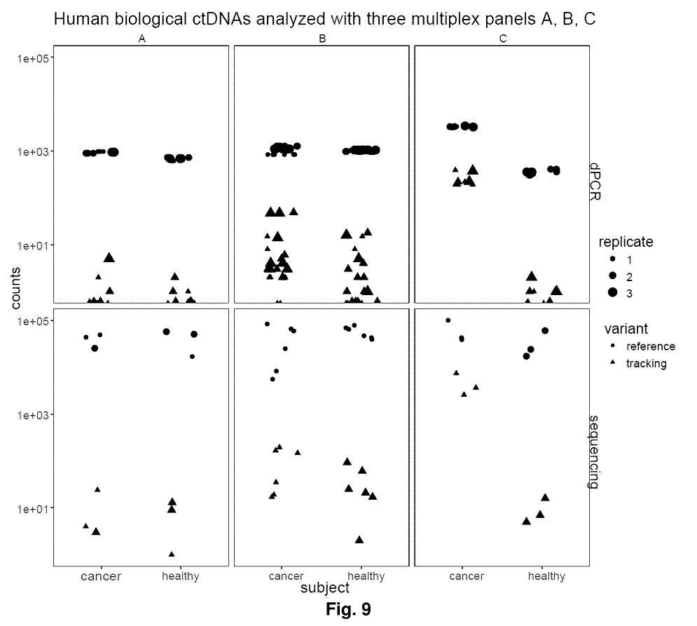

Plasma and matched CRC tumor sequencing data are obtained from three human cancer patients (A, B, and C). Consents are collected and experiments are carried out in accordance with industry standards and clinical protocols. Additional plasma samples from healthy human subjects were obtained for comparison. Cell free DNA (cfDNA) is isolated from all plasma samples using commercially available isolation kits. Tumor-specific dPCR assays based on each patient's tumor sequencing profile are designed and assembled to measure the “tracking variants” and run on isolated cfDNA from each plasma sample. In parallel, a targeted sequencing assay (“NGS”) is run on the same cfDNA samples. The result is shown in FIG. 9. A wild-type “reference” target was measured in parallel as a positive control.

### Example 5

### In vitro Multiplex Assay Capable of Simultaneously Detecting 26 Different Cancer Variants

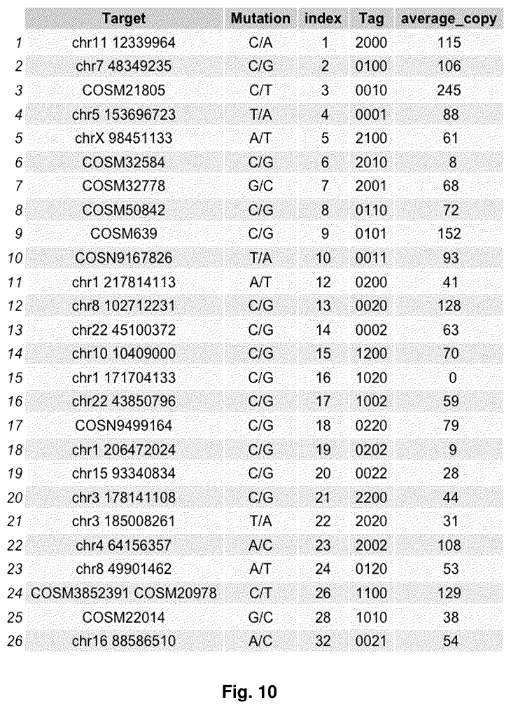

Three cancer cell lines (HCC1395, HCC1143, and HCC1187) are combined and used to inform the construction of a 26-target digital PCR assay. The cancer cell line mixture is titrated in a background of wild type genomic DNA (NA12878). The first position in mutation indicates the reference target allele genotype and the second position indicates the mutation present and the allele being detected (Reference/Alternate). The index column indicates which of the tags has been assigned to the target. All 26 targets were detected simultaneously in a single reaction at a 12.5% dilution, with each target having a unique tag sequence (see FIG. 10). The mean number of copies for each target is described in the “average_copy” column (3 replicates).

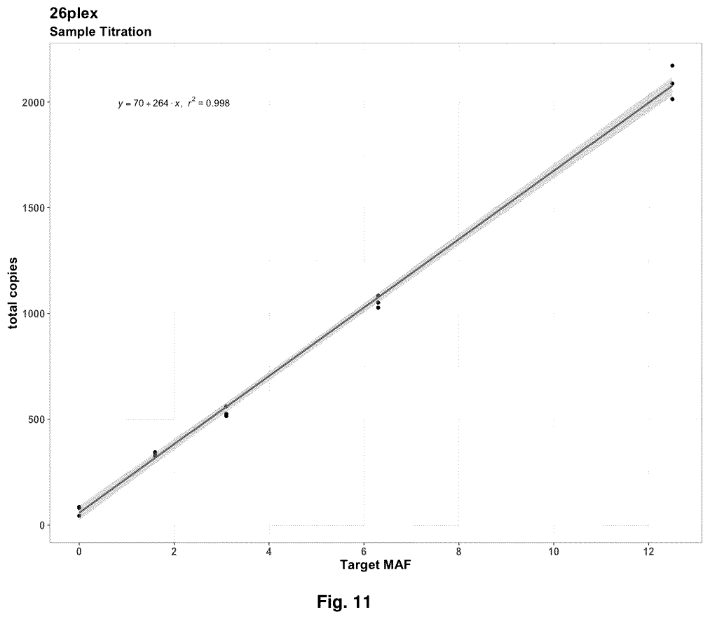

A 2-fold dilution series of the cancer cell line mixtures from FIG. 10 are tested and the results shown in FIG. 11. The Y-axis reflects the cumulative copy number detected for all 26 targets. The assay illustrates linear sensitivity with an r²=0.998.

### Example 6

### Unambiguous Resolving the Targets

With the universal signal encoding scheme we have designed, some partitions at the end of a dPCR reaction can yield a signal, which is ambiguous as to which genetic targets are present in that partition. For example, if three of the targets are given signals 1000, 0100, and 1100, then a partition with a final signal reading of 1100 could potentially have five different target combinations present: (a) 1100, (b) 1000+0100, (c) 1100+1000, (d) 1100+0100, or (e) 1100+1000+0100.

To calculate a final value for the number of copies of each target present in the original sample, statistics can be used to compute the relative probabilities of these various possible target compositions. Assuming that Poisson statistics are applicable (i.e., that all DNA molecules in the original sample are distributed randomly, uniformly, and independently between the dPCR partitions), the targets can be analyzed to resolve the ambiguity.

Using a 4-channel signal encoding scheme as an example, the analytic process can be as follows:

First, every partition is assigned a signal based on its amplitude in each color channel compared to calibrator intensities. This uniquely determines which universal probe sequences is amplified in each partition, however, may leave ambiguity between different amplified target combinations that could have contributed those universal probe sequences.

Second, every partition with a signal of 0000 is known to contain no amplified targets, so the number of 0000 partitions provides a “null count”.

Third, partitions with a signal corresponding to a target with a probe in only one-color channel (e.g., 1000 or 0100) are known to contain exactly that single target and no others. Based on the ratio between the number of these single-channel partitions to the null count, the number of copies of the corresponding single-channel targets must be present can be computed.

Fourth, for partitions with a signal corresponding to a target with a probe in exactly two-color channels (e.g., 1100), which may contain various different target combinations. From the third step above, the concentration of all possible single-channel component targets (in this case, 1000 and 0100) can be identified. Using these concentrations and knowledge of all possible target combinations which can produce a 1100 partition, the number of copies of the 1100 target that must be present such that all of the various combinations with the 1000 and 0100 targets add up to the proper number of 1100 partitions can be computed.

Five, once the fourth step above has been performed for all targets with 2-channel signals, the analysis can be repeated for all targets with 3-channel signals, and then all targets with 4-channel signals to resolve all ambiguities and compute how many copies of each target are present in the original sample.

The example above is provided for a four-channel signal, but the method can be applied to any scheme across any number of color channels.

### REFERENCES

The following publications, references, patents, and patent applications are hereby incorporated by reference in their entireties.
Schwartz J et al., Methods, Systems and Compositions for Detection of Multiple Analytes, PCT/US2023/070277, published as WO2024015999A1.
Rajagopal, A et al., Nucleic Acid Reactions and Related Methods and Compositions. US20190032112A1.
Rajagopal, A et al., Nucleic acid reactions and related methods and compositions. US20230038055A1.
Abbosh, C et al., Phylogenetic ctDNA analysis depicts early-stage lung cancer evolution. Nature, 2017; 545(7655), 446-451.
Kotani, D et al., Molecular residual disease and efficacy of adjuvant chemotherapy in patients with colorectal cancer. Nat Med. 2023; 29 127-134.
Flach S et al., et al. Liquid BIOpsy for MiNimal RESidual Disease Detection in Head and Neck Squamous Cell Carcinoma (LIONESS)-a personalised circulating tumour DNA analysis in head and neck squamous cell carcinoma. British Journal of Cancer 2022; 126(8):1186-1195.
Lipsyc-Sharf M et al., Survival in young-onset metastatic colorectal cancer: findings from cancer and leukemia group B (Alliance)/SWOG 80405. J Natl Cancer Inst. 2022; 114(3):427-435.
Kurtz D M et al., Enhanced detection of residual disease by targeted sequencing of phased variants in circulating tumor DNA. Nature Biotechnology. 2021 December; 39(12):1537-1547.

What is claimed is:

1. A composition for use in a patient-specific PCR, the composition comprising:
(a) a set of primers each encoding a sequence complementary to a unique patient-specific tumor variant sequence and
(b) a DNA polymerase; and/or
(c) a RNase H; and/or
(d) a dNTP mixture; and/or
(e) a buffer; and/or
(f) a magnesium compound; and/or;
wherein the composition further comprises (g) a set of detection probes;
wherein each detection probe comprises:
a fluorophore and optionally a quencher,
wherein the fluorophore and optionally the quencher is conjugated to the probe;
wherein each detection probe encodes a sequence complementary to the primer; and
wherein the patent specific tumor variant sequence correlates with or is associated with a lesion, benign tumor, pre-malignant tumor, malignant tumor, neoplasia, dysplasia, hyperplasia, hamartoma, and/or other pre-cancerous and cancerous conditions with abnormal cell growth in the patient.

2. The composition of claim 1, wherein the patent specific tumor variant sequence is different from the corresponding sequence of a normal somatic cell in the patient.

3. The composition of claim 1, wherein the number of unique detection probes for each unique patient-specific tumor variant sequence is between 1 and nₚ, wherein np is selected from the list consisting of 1, 2, 3, 4, 5, 6, 7, 8, 9, 10, 11, and 12

4. The composition of claim 1, wherein the number of unique detection probes specific for the unique patient-specific tumor variant sequences in a sample volume is between 1 and Nₚ, wherein Nₚ is selected from the list of 1, 4, 8, 12, 16, 20, 24, 28, 32, 36, 40, 44, 48, 52, 56, 60, 64, 68, 72, 76, 80, 84, 88, 92 and 96 or 2, 3, 4, 5, 6, 7, 8, 9, 10, 11, 12, 13, 14, 15, 16, 17, 18, 19, and 20.

5. The composition of claim 1, wherein the first detection probe is conjugated to a first fluorophore and optionally conjugated to a first quencher.

6. The composition of claim 1, wherein the combination of the emission color and emission intensity for each detection probe is unique.

7. The composition of claim 1,
wherein the fluorophore is selected from a group of ABY, Alexa Fluor 350, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750, ATTO 425, ATTO 550, ATTO 590, Cyan500, Cy3, Cy5, Cy5.5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, VIC, LC610, CFR610, JA270, LC640, JUN, Trimethylrhodamine (TRITC), Cal Fluor dyes, Quasar dyes, DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), derivatives thereof and combinations thereof; and
wherein the quencher is selected from the group consisting of TAMRA, BHQ-1, BHQ-2, BHQ-3, IowaBlack FQ, ZEN, or Dabcy, derivatives thereof, and combinations thereof.

8. The composition of claim 1,
wherein the set of primers each encode a set of tag sequences, and
wherein the number of unique tag sequence on any individual primer is between 1 and n, wherein n is selected from the list consisting of 1, 2, 3, 4, 5, 6, 7, 8, 9, 10, 11, and 12.

9. The composition of claim 1,
wherein the set of primers each encode a set of tag sequences, and
wherein the total number of unique tag sequences on the set of primers is between 1 and N, wherein N is selected from the list consisting of 4, 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536.

10. The composition of claim 1,
wherein the set of primers each encode a set of tag sequences, and
wherein the total number of unique primers in the set of primers is between 1 and X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, 1536, 1540, 1544, 1548, 1552, 1556, 1560, 1564, 1568, 1572, 1576, 1580, 1584, 1588, 1592, 1596, 1600, 1604, 1608, 1612, 1616, 1620, 1624, 1628, 1632, 1636, 1640, 1644, 1648, 1652, 1656, 1660, 1664, 1668, 1672, 1676, 1680, 1684, 1688, 1692, 1696, 1700, 1704, 1708, 1712, 1716, 1720, 1724, 1728, 1732, 1736, 1740, 1744, 1748, 1752, 1756, 1760, 1764, 1768, 1772, 1776, 1780, 1784, 1788, 1792, 1796, 1800, 1804, 1808, 1812, 1816, 1820, 1824, 1828, 1832, 1836, 1840, 1844, 1848, 1852, 1856, 1860, 1864, 1868, 1872, 1876, 1880, 1884, 1888, 1892, 1896, 1900, 1904, 1908, 1912, 1916, 1920, 1924, 1928, 1932, 1936, 1940, 1944, 1948, 1952, 1956, 1960, 1964, 1968, 1972, 1976, 1980, 1984, 1988, 1992, 1996, 2000, 2004, 2008, 2012, 2016, 2020, 2024, 2028, 2032, 2036, 2040, 2044, 2048, 2052, 2056, 2060, 2064, 2068, 2072, 2076, 2080, 2084, 2088, 2092, 2096, 2100, 2104, 2108, 2112, 2116, 2120, 2124, 2128, 2132, 2136, 2140, 2144, 2148, 2152, 2156, 2160, 2164, 2168, 2172, 2176, 2180, 2184, 2188, 2192, 2196, 2200, 2204, 2208, 2212, 2216, 2220, 2224, 2228, 2232, 2236, 2240, 2244, 2248, 2252, 2256, 2260, 2264, 2268, 2272, 2276, 2280, 2284, 2288, 2292, 2296, 2300, 2304, 2308, 2312, 2316, 2320, 2324, 2328, 2332, 2336, 2340, 2344, 2348, 2352, 2356, 2360, 2364, 2368, 2372, 2376, 2380, 2384, 2388, 2392, 2396, 2400, 2404, 2408, 2412, 2416, 2420, 2424, 2428, 2432, 2436, 2440, 2444, 2448, 2452, 2456, 2460, 2464, 2468, 2472, 2476, 2480, 2484, 2488, 2492, 2496, 2500, 2504, 2508, 2512, 2516, 2520, 2524, 2528, 2532, 2536, 2540, 2544, 2548, 2552, 2556, 2560, 2564, 2568, 2572, 2576, 2580, 2584, 2588, 2592, 2596, 2600, 2604, 2608, 2612, 2616, 2620, 2624, 2628, 2632, 2636, 2640, 2644, 2648, 2652, 2656, 2660, 2664, 2668, 2672, 2676, 2680, 2684, 2688, 2692, 2696, 2700, 2704, 2708, 2712, 2716, 2720, 2724, 2728, 2732, 2736, 2740, 2744, 2748, 2752, 2756, 2760, 2764, 2768, 2772, 2776, 2780, 2784, 2788, 2792, 2796, 2800, 2804, 2808, 2812, 2816, 2820, 2824, 2828, 2832, 2836, 2840, 2844, 2848, 2852, 2856, 2860, 2864, 2868, 2872, 2876, 2880, 2884, 2888, 2892, 2896, 2900, 2904, 2908, 2912, 2916, 2920, 2924, 2928, 2932, 2936, 2940, 2944, 2948, 2952, 2956, 2960, 2964, 2968, 2972, 2976, 2980, 2984, 2988, 2992, 2996, 3000, 3004, 3008, 3012, 3016, 3020, 3024, 3028, 3032, 3036, 3040, 3044, 3048, 3052, 3056, 3060, 3064, 3068, and 3072; and/or

11. The composition of claim 1,
wherein the set of primers each encode a set of tag sequences, and
wherein the number of unique patient-specific tumor variant sequence is from 1 to X, wherein X is selected from the list consisting of 8, 12, 16, 20, 24, 28, 32, 36, 40, 44, 48, 52, 56, 60, 64, 68, 72, 76, 80, 84, 88, 92, 96, 100, 104, 108, 112, 116, 120, 124, 128, 132, 136, 140, 144, 148, 152, 156, 160, 164, 168, 172, 176, 180, 184, 188, 192, 196, 200, 204, 208, 212, 216, 220, 224, 228, 232, 236, 240, 244, 248, 252, 256, 260, 264, 268, 272, 276, 280, 284, 288, 292, 296, 300, 304, 308, 312, 316, 320, 324, 328, 332, 336, 340, 344, 348, 352, 356, 360, 364, 368, 372, 376, 380, 384, 388, 392, 396, 400, 404, 408, 412, 416, 420, 424, 428, 432, 436, 440, 444, 448, 452, 456, 460, 464, 468, 472, 476, 480, 484, 488, 492, 496, 500, 504, 508, 512, 516, 520, 524, 528, 532, 536, 540, 544, 548, 552, 556, 560, 564, 568, 572, 576, 580, 584, 588, 592, 596, 600, 604, 608, 612, 616, 620, 624, 628, 632, 636, 640, 644, 648, 652, 656, 660, 664, 668, 672, 676, 680, 684, 688, 692, 696, 700, 704, 708, 712, 716, 720, 724, 728, 732, 736, 740, 744, 748, 752, 756, 760, 764, 768, 772, 776, 780, 784, 788, 792, 796, 800, 804, 808, 812, 816, 820, 824, 828, 832, 836, 840, 844, 848, 852, 856, 860, 864, 868, 872, 876, 880, 884, 888, 892, 896, 900, 904, 908, 912, 916, 920, 924, 928, 932, 936, 940, 944, 948, 952, 956, 960, 964, 968, 972, 976, 980, 984, 988, 992, 996, 1000, 1004, 1008, 1012, 1016, 1020, 1024, 1028, 1032, 1036, 1040, 1044, 1048, 1052, 1056, 1060, 1064, 1068, 1072, 1076, 1080, 1084, 1088, 1092, 1096, 1100, 1104, 1108, 1112, 1116, 1120, 1124, 1128, 1132, 1136, 1140, 1144, 1148, 1152, 1156, 1160, 1164, 1168, 1172, 1176, 1180, 1184, 1188, 1192, 1196, 1200, 1204, 1208, 1212, 1216, 1220, 1224, 1228, 1232, 1236, 1240, 1244, 1248, 1252, 1256, 1260, 1264, 1268, 1272, 1276, 1280, 1284, 1288, 1292, 1296, 1300, 1304, 1308, 1312, 1316, 1320, 1324, 1328, 1332, 1336, 1340, 1344, 1348, 1352, 1356, 1360, 1364, 1368, 1372, 1376, 1380, 1384, 1388, 1392, 1396, 1400, 1404, 1408, 1412, 1416, 1420, 1424, 1428, 1432, 1436, 1440, 1444, 1448, 1452, 1456, 1460, 1464, 1468, 1472, 1476, 1480, 1484, 1488, 1492, 1496, 1500, 1504, 1508, 1512, 1516, 1520, 1524, 1528, 1532, and 1536.

12. The composition of claim 1, wherein the patent specific tumor variant sequence is different from the corresponding sequence of a normal somatic cell in the patient.

13. The composition of claim 1, further comprising a DNA probe sequence,
wherein the DNA probe sequence comprise 11 to 30 nucleotide bases, and
wherein the DNA probe sequence exhibiting a melting point between 45° C. and 75° C.

14. The composition of claim 13, wherein the DNA probe sequence is either unmodified or modified to achieve a melting point between 65° C. and 75° C. by:
including one or more locked nucleic acid (LNA) bases and/or;
including one or more peptide nucleic acid (PNA) bases and/or;
including one or more 2′-O-methyl RNA nucleotides and/or;
including one or more phosphorothioate (PS) linkage modifications and/or;
further conjugating with minor groove binding (MGB) proteins.

15. The composition of claim 13, wherein each detection probe encodes a sequence complementary to a synthetic sequence encoded by a primer; and
wherein composition emit a unique signal in the sequence encoded by the primer.

16. A method of making the composition of claim 1 for patient-specific PCR, the method comprising:
(a) isolating
a first genomic DNA from a first sample of a patient and
a second genomic DNA from a second sample of the patient,
(b) sequencing
the first genomic DNA and
the second genomic DNA,
(c) identifying a set of the unique patient-specific tumor variant sequences
(d) synthesizing the set of primers for the patient-specific tumor variant sequences identified in step (c), and
(e) combining the set of primers with the set of reagents to form the composition for patient-specific PCR.

17. The method of claim 16, wherein the patient-specific PCR is used to detect, analyze, evaluate, screen for, prognose, diagnose, and/or monitor, a condition in the patient; and/or
wherein the condition is optionally
a lesion, benign tumor, pre-malignant tumor, malignant tumor, neoplasia, dysplasia, hyperplasia, hamartoma, and/or other pre-cancerous and cancerous conditions with abnormal cell growth.

18. The method of claim 16,
wherein the first sample comprises
a cancerous tissue biopsy and/or
a tissue biopsy suspected of being cancerous; and/or
wherein the second sample comprises
a normal or non-cancerous blood sample, preferably a normal buffy coat of the blood sample and/or
a normal or non-cancerous tissue biopsy; and/or
wherein the first genomic DNA comprise
DNA that is cancerous and/or
DNA that is suspected of being cancerous; and/or
wherein the second genomic DNA comprises
a normal DNA, preferably isolated from a leukocytes or a buffy coat of the second sample.

19. The method of claim 16, wherein the sequencing of step (b):
is not whole genome sequencing.

20. The method of claim 16, wherein the sequencing of step (b) comprises
exome sequencing; and/or
deep targeted sequencing; and/or
shearing the genomic DNA from the first sample and/or the second sample into fragments having a length of from approximately 2 to 2000 nucleotides, from 2 to 4000 nucleotides, from 2 to 10,000 nucleotides.
